(19) United States
(12) Patent Application Publication
(10) Pub. No.: US 2011/0021440 A1
(43) Pub. Date: Jan. 27, 2011

(54) APOPTOTIC PATHWAY TARGETING FOR THE DIAGNOSIS AND TREATMENT OF CANCER

(75) Inventors: Stuart S. Martin, Severna Park, MD (US); Anges Cheung, Arnold, MD (US); Rebecca Whipple-Bettes, Elkridge, MD (US)

Correspondence Address: SUGHRUE MION, PLLC, 2100 PENNSYLVANIA AVENUE, N.W., SUITE 800, WASHINGTON, DC, 20037, US

(73) Assignee: UNIVERSITY OF MARYLAND, BALTIMORE, Baltimore, MD (US)

(21) Appl. No.: 12/599,784

(22) PCT Filed: May 16, 2008
(86) PCT No.: PCT/US08/63892

§ 371 Date: Oct. 4, 2010

Related U.S. Application Data
(60) Provisional application No. 60/938,224, filed on May 16, 2007.

(51) Int. Cl. A61K 38/16; C12N 5/09; A61P 35/00

(52) U.S. Cl. 514/19.3; 435/325

(57) ABSTRACT

The invention relates to methods of treating cancer. The invention further relates to a method of treating cancer by exploiting apoptotic pathways. The invention particularly relates to regulation of apoptotic pathways in cancerous cells, to metastasis of cancer cells, and to methods of preventing cancer metastasis.

### CROSS-REFERENCE TO RELATED APPLICATION

This application claims benefit of U.S. Provisional Application No. 60/938,224, filed May 16, 2007; the disclosure of which is incorporated herein by reference.

### STATEMENT OF FEDERALLY SPONSORED RESEARCH AND DEVELOPMENT

This work was supported, in part by, the National Institutes of Health, Grant Nos. K01-CA096555 and S10-RR022434-01; Department of Defense Congressionally Directed Medical Research Programs Breast Cancer Concept Award, Grant No. BC045699 and an Independent New Investigator award from the University of Maryland, Baltimore. The United States Government has certain rights in the invention.

### TECHNICAL FIELD

The invention relates to cancer biology. The invention further relates to regulation of apoptotic pathways in cancerous cells. The invention also relates to metastasis of cancer cells and methods of diagnosing, preventing, and treating the same.

### BACKGROUND OF INVENTION

### Cancer

Cancer imposes a tremendous burden on society from the standpoint of both pain and suffering and economics. Cancer cells generally grow unregulated and alter normal cell or organ function at the site of growth or some distal site. Loss of p53 function is one of the most common mutations in human cancer (Royds, Cell Death Differ., 13:1017-1026 (2006)). Inhibition of p53 function occurs via numerous mechanisms, including direct mutation of the p53 coding sequence, methylation of its promoter, misregulated p53 degradation, and loss of heterozygosity (Id.). Clearly disabling p53 provides a strong selective advantage to tumors, since this single alteration can simultaneously impact regulation of the cell cycle, genetic stability and apoptosis (Id.). However, overexpression of the antiapoptotic Bcl-2 protein can remove the selective pressure to mutate p53 (Gurova, Cancer Biol. Ther., 1:39-44 (2002)). This may explain why there are such contrasting clinical results concerning antiapoptotic proteins like Bcl-2.

A successful cure for breast cancer will require the destruction of tumor cells that spread throughout the body. Breast tumor cells that are circulating in the bloodstream can invade distant tissues and lie dormant for long periods (Naumov et al., Cancer Res., 62:2162-2168 (2002); Naumov et al., Semin Cancer Biol., 11:271-276 (2001); and Schmidt-Kittler et al, Proc. Natl. Acad. Sci., U.S.A., 100:7737-7742 (2003). The eventual reemergence of these disseminated cells as metastatic tumors is a major cause of patient death (Chambers et al., Nat. Rev. Cancer, 2:563-572 (2002). Our lab's research has shown that apoptotic resistance promotes tumor dormancy by allowing cells to survive the challenges of bloodborne dissemination, but failing to initiate active tumor growth (Martin et al., Mol. Cell. Biol., 21:6529-6536 (2001); Martin et al., Oncogene, 23:4641-4645 (2004); and Pinkas et al., Mol. Cancer. Res., 2:551-556 (2004)). Such dormant cells persist without active cell division and are therefore resistant to many traditional chemotherapies (Naumov et al, Breast Cancer Res. Treat., 82:199-206 (2003)). Destroying these dormant tumor cells is critical to prevent metastatic recurrence, since their presence predicts poor patient outcome in breast cancer (Klein et al., Lancet, 360:683-689 (2002).

Tumor cells can die by apoptosis when leaving the primary tumor site, as a result of detachment from the extracellular matrix in the originating tissue and the resulting rounding of the cells. Since large epithelial tumor cells are trapped efficiently in the first capillary bed encountered, tumor cells that resist apoptosis will survive the transit to this distant tissue. For most tissues in the body, the first capillary bed encountered is in the lung. An exception to this is intestinal tumor cells, which drain first through the portal vein into the liver. Circulatory patterns can therefore dictate the organ most likely to pose a risk for metastatic tumor recurrence. Studies have shown that once trapped, tumor cells often escape the capillary into adjacent tissue within 24 hours (Chambers et al, Nat. Rev. Cancer, 2:563-572 (2002)). If the tumor cells fail to escape the vessel, they will often be crushed as blood pressure pushes them through the narrow capillary (Morris et al, Clin. Exp. Metastasis, 11:377-390 (1993); and Tsuji et al., Cancer Res., 66:303-306 (2006)). Once these apoptotically-resistant tumor cells colonize the distant tissue, they often enter a period of long-term dormancy (1-5 years) before entering a recurrent growth phase as metastatic tumors. Detection of such disseminated cells in human patients is a powerful predictor of patient death, indicating that these cells are the likely source of metastatic recurrence (Klein et al., Lancet, 360:683-689 (2002)).

Tumor cells which resist apoptosis can persist dormantly, but pose some significant challenges for therapy. Continued activation of p53 leads to long-term cell cycle arrest (Nikiforov et al., Oncogene, 13:1709-1719 (1996)), and resistance to traditional chemotherapies (Naumov et al., Breast Cancer Res. Treat., 82:199-206 (2003)).

### Apoptosis

Apoptosis, also known as programmed cell death, is a physiological response to rid the body of unneeded or undesirable native cells. The process of apoptosis is used during development to remove cells from areas where they are no longer required, such as the interior of blood vessels or the space between digits. Apoptosis is also important in the body's response to disease. Cells that are infected with some viruses can be stimulated to undergo apoptosis, thus preventing further replication of the virus in the host organism.

Impaired apoptosis due to blockade of the cell death-signaling pathways is involved in tumor initiation and progression, since apoptosis normally eliminates cells with increased malignant potential such as those with damaged DNA or aberrant cell cycling (White, Genes Dev., 10:1-15 (1996)).

Cancer cells are protected by at least one of two anti-apoptotic factors, Bcl-2 or Bcl-xL. Members of the Bcl-2-family are known to modulate apoptosis in different cell types in response to various stimuli. Some members of the family act to inhibit apoptosis, such as Bcl-2 and Bcl-xL, while others, such as BAX, BAK, Bid, and Bad promote apoptosis. The ratio at which these proteins are expressed can dictate whether a cell undergoes apoptosis or not. For instance, if the Bcl-2 level is higher than the BAX level, apoptosis is suppressed. If the opposite is true, apoptosis is promoted. Bcl-2 and Bcl-xL overexpression contributes to cancer cell progression by preventing normal cell turnover caused by physiological programmed cell death mechanisms, and has been observed in a number of cancers (Reed, Sem. Hematol., 34:9-19 (1997); Buolamwini, Curr. Opin. Chem. Biol., 3:500-509 (1999); Espana, Breast Cancer Research and Treatment, 87: 33-44 (2004)).

Damage or stress on epithelial cells, such as that induced by cell rounding, leads to activation of the p53 tumor suppressor protein which imposes cell cycle arrest through proteins such as p21 (CIP1/WAF1). If the stress persists, p53 alters Bcl-2 family proteins to induce apoptotic cell death, primarily by upregulating levels of the pro-apoptotic Bcl-2 protein, Bax. Overexpression of pro-survival Bcl-2 proteins, such as Bcl-2 or Bcl-xL can rescue cell death, but will not prevent the ability of p53 to arrest the cell cycle, leading to tumor dormancy (Nikiforov et al., Oncogene, 13:1709-1719 (1996)). However, inactivating mutations or downregulation of p53 protein is one of the most common events in human solid tumors, occurring in approximately 50-60% of cancer patients. Once p53 function is lost, the selective pressure to alter any of the Bcl-2 proteins or other apoptotic regulators through expression or mutation is removed, since the p53 loss-of-function eliminates both the cell cycle and apoptotic controls. In order to identify novel proteins that might influence apoptotic signaling in human tumors, it is essential to determine whether p53 signaling remains intact. It would be helpful to determine p53 functionality through microarray expression analysis, which is being conducted with greater frequency in patients. However, p53 is regulated largely at the post-translational level, so levels of p53 mRNA are not indicative of p53 function.

### BRIEF SUMMARY OF INVENTION

Loss of p53 function is one of the most common mutations in human cancer (Royds, Cell Death Differ., 13:1017-1026 (2006)). Numerous posttranscriptional mechanisms regulate p53 activity so gene expression data cannot simply be examined for levels of p53 mRNA to determine if the pathway remains intact. A novel and more effective approach is to identify transcriptional targets of p53 that could serve as indicators of p53 function and allow filtration of patient gene expression data on the basis of an intact p53 pathway. This approach is used to identify patient samples with transcriptional indicators of positive p53 function to isolate novel indicators of diagnosis, prognosis, and therapeutic targets for cancer.

The invention relates to methods of treating cancer. The invention also relates to methods of screening for prognostic markers of cancer and therapeutic targets for the treatment of cancer. The invention also relates to diagnosing cancer. The invention also relates to the treatment and prevention of cancer metastasis.

In particular embodiments, the invention is drawn to a method of treating cancer in a subject in need of such treatment comprising the administration to said subject of an effective dose of an apoptosis inducing agent. In other embodiments, the apoptosis inducing agent is selected from an agent that is a member of the Bcl-2 family, or a derivative thereof, such as, for example the group consisting of a Bcl-2 inhibitor and a Bcl-xL inhibitor. In other embodiments, the apoptosis inducing agent of the invention is administered in combination with one or more methods of treating cancer selected from the group consisting of administering an anticancer agent, radiation therapy, and surgical therapy. In specific embodiments, the apoptosis inducing agent is administered in combination with surgical therapy. In other specific embodiments, the apoptosis inducing agent is administered in advance of surgical therapy. In other specific embodiments, the surgical therapy is surgical tumor resection.

In particular embodiments, the invention is drawn to a method of killing a cancer cell comprising contacting said cancer cell with an effective dose of an apoptosis inducing agent. Apoptosis can be overt or cellular stress. In other embodiments, the apoptosis inducing agent is selected from an agent that is a member of the Bcl-2 family, or a derivative thereof, such as, for example the group consisting of a Bcl-2 inhibitor and a Bcl-xL inhibitor. In other embodiments, contacting the cancer cell with an effective dose of an apoptosis inducing agent is carried out in combination with one or more anticancer agents or radiation therapy.

In particular embodiments, the invention is drawn to a method of preventing tumor metastasis in a subject in need of such prevention comprising the administration to said subject of an apoptosis inducing agent. In other embodiments, the apoptosis inducing agent is selected from an agent that is an inhibitor of a member of the Bcl-2 family, or a derivative thereof, such as, for example the group consisting of a Bcl-2 inhibitor and a Bcl-xL inhibitor. In other embodiments, the apoptosis inducing agent is selected from the group consisting of Bak NM_{—}001188, Bmf NM_{—}001003940, Bik NM_{—}001197, Bid NM_{—}197966, Bad NM_{—}004322, Bim AF032458, Bcl-2 NM_{—}000633, and Bcl-xL NM_{—}138578, or combinations thereof. In other embodiments, the apoptosis inducing agent of the invention is administered in combination with one or more methods of treating cancer selected from the group consisting of administering an anticancer agent, radiation therapy, and surgical therapy. In specific embodiments, the apoptosis inducing agent is administered in combination with surgical therapy. In other specific embodiments, the apoptosis inducing agent is administered in advance of surgical therapy. In other specific embodiments, the surgical therapy is surgical tumor resection.

In particular embodiments, the invention is drawn to a method of analyzing a sample utilizing gene expression data related to cancer using a filter mechanism whereby said mechanism uses a first gene as an indicator of the function of a second gene, for example, a gene related to cancer, allowing for distinguishing a sample as functional or non-functional (i.e., loss of function) for the second gene and a functional pathway associated therewith. A second gene and pathway associated therewith can be determined to be functional or non-functional by comparing a first gene serving as an indicator to a control that correlates the first gene serving as an indicator to the second gene and pathway associated therewith as functional or non-functional. A control can, for example, be established by quantifying varying known amounts of a first gene that is indicative of whether or not a second gene and pathway associated therewith is functional or non-functional.

In other embodiments, the invention is drawn to analyzing the distinguished samples to determine prognostic indicators related to cancer. In specific embodiments, the first gene as an indicator of the function of a second gene is Reprimo, GADD45, or both Reprimo and GADD45. In other specific embodiments, the second gene for which Reprimo, GADD45, or both Reprimo and GADD45 indicate function is p53 and a functional pathway associated therewith. In other specific embodiments the first gene as an indicator of the function of a second gene is SCN3B NM_{—}018400, Reprimo NM_{—}019845, p21 NM_{—}000389, MDM2 NM_{—}006878, GADD45g NM_{—}006705, ADD45a NM_{—}001924, Noxa NM_{—}021127, PUMA AF354654, PERP NM_{—}022121, Siah-1 NM_{—}003031, TSP-1 NM_{—}003246, DDB2 NM_{—}000107, IGF-BP3 NM_{—}001013398, Killer/DR5 NM_{—}147187, PIG-3 BC000474, Fas NM_{—}000043, Bax NM_{—}138761, or any combination thereof.

The foregoing teaches the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1 illustrates Bik expression is regulated by the proteasome and upregulated by cell rounding in MCF10A cells. FIG. 1A relates to human mammary epithelial cells (MCF10A) grown for 24 h in the presence or absence of a proteasomal inhibitor (MG132, 5 μM). Western blotting indicates that Bik protein is strongly upregulated by proteasomal inhibition. Lysates from Ramos cells are included as a positive control for Bik and parallel blotting for β-actin confirm equivalent levels of protein. FIG. 1B relates to Bik protein increases after forced cell rounding with the actin polymerization inhibitor, Latrunculin-A (LA, 5 μM for 24 h). Bik protein is low in standard growth media (GROW). Additional apoptotic stimuli such as serum starvation (DMEM), TRAIL (1 μg/mL), Cycloheximide (CHX, 30 μg/mL) or Doxorubicin (DOX, 3 μg/mL) do not increase Bik levels, even though each treatment causes cell death comparable to LA in MCF10A cells (not shown). Cell rounding may therefore act selectively to upregulate Bik rather than through a nonspecific apoptotic signal.

FIG. 2 illustrates Bik expression is strongly upregulated by the presence of Bcl-2 or Bcl-xL. Western blotting shows increased Bik protein in MCF10A cell lines that stably express the survival protein Bcl-2, even under standard growth conditions (Control). MCF10A or MCF-7 cells show comparably low levels of Bik. Forced cell rounding with Latrunculin-A (LA) induces dramatic increases in Bik protein in cell lines expressing the survival proteins Bcl-2 or Bcl-xL. This increase in Bik is so strong that the moderate increases observed with LA in MCF10A cells (FIG. 7B) cannot even be detected here due to the short exposure times necessary to prevent oversaturating signal.

FIG. 3 illustrates p53 inducible genes.

FIG. 4 illustrates that Reprimo and GADD45 as indicators of p53 function identify the prognostic significance of Bik. FIG. 4A relates to Raw Bik expression data showing no significant difference between patients of different prognosis groups. FIG. 4B shows the differences in the average Bik mRNA expression profiles between prognosis groups were not statistically significant in the raw data, but were strongly significant once expression data was filtered relative to either Reprimo expression alone or the combination of Reprimo and GADD45.

FIG. 5 shows Bik mRNA is reduced in human breast tumor cell lines. FIG. 5A shows real-time PCR of Bik mRNA in MCF10A nontumorigenic human mammary epithelial cells or 11 breast tumor cell lines. FIG. 5B shows amplification curves for Bik show varying levels of mRNA in tumor samples, but consistent amplification of GAPDH. Sybr-green dissociation curves confirm that a single major product resulted from each amplification.

FIG. 6 illustrates Bik protein is degraded by the proteasome but remains reduced in tumor cell lines. Protein lysates from control cells (Ramos), nontumorigenic human mammary epithelial cells (MCF10A) or 11 human breast tumor cell lines were immunoblotted for Bik and b-actin as a loading control. While Bik protein was very low in untreated MCF10A samples (−MG132), inhibition of proteasomal degradation (+MG132, 5 μM−24 h) strongly increased Bik protein. Bik protein remained low in the tumor cell lines, even in the presence of MG132.

FIG. 7 illustrates tumor lines with reduced Bik resist apoptosis by cytoskeletal disruption. FIG. 7A shows in MCF10A cells, Bik is normally expressed at very low levels (Growth) compared to a positive control cell lysate (Ramos). Apoptotic induction by 24 hours of either serum deprivation (DMEM), TRAIL (1 μg/mL), cycloheximide (CHX, 30 μg/mL) or Doxorubicin (3 μg/mL) did not increase Bik protein. Actin disruption with Latrunculin-A (LA, 5 μM) did increase Bik significantly, as compared to an α-tubulin loading control. FIG. 7B shows cell viability assay (XTT) of MCF10A cells and tumor cell lines were treated with the indicated concentrations of LA. FIG. 7C shows cell viability values at 5 μM LA demonstrate the significant resistance to apoptosis by cell rounding in the tumor cell lines with reduced Bik expression.

FIG. 8 illustrates long-term survival of circulating mammary epithelial cells. NCR-nu/nu mice were injected via the tail vein with either 1×10⁶ luciferase-expressing EpH4 cells (EpLuc) or 1×10⁶ EpH4-Bcl2 cells (BLuc). FIG. 8A shows immediately following injection, mice were injected with D-Luciferin (150 mg/kg), anesthetized with 2.5% isoflurane and imaged for 5 minutes on a Xenogen IVIS-200 optical imaging system with 4×4 pixel binning for high sensitivity. Efficient trapping of circulating tumor cells in the lungs is evident (black arrows). Mice were then imaged at time points of 1, 2, 5, 10, 20 (shown) and 50 days. FIG. 8B shows bioluminescence in each mouse was normalized to the initial recording following injection to correct for differences in injection efficiency. Survival of BLuc cells is 367% higher than EpLuc cells at 50 days (P<0.02, t-test)*, but a substantial percentage (37%) of EpLuc cells also persist. Neither cell line will produce tumors after even 12 weeks. Each point represents the mean+/−S.E.M. of bioluminescence readings taken from three individual mice, with background luminescence values from uninjected mice subtracted. Although apoptotic resistance improves dormant tumor cell survival, the opportunity for independent evolution of primary and metastatic tumors may be greater than previously appreciated, since many EpLuc cells also survive long-term in distant tissues.

FIG. 9 illustrates that apoptotic resistance promotes tumor dormancy and metastatic recurrence. Dormant cells also upregulate the drug resistance protein BCRP when rounded (FIG. 12) and that apoptotically-resistant cells produce tubulin microtentacles for extended period of time, making them more invasive and likely to adhere in distant tissues (Whipple et al., Exp. Cell Res., 313:1326-1336 (2007)). However, current data also indicate that these rounded cells demonstrate persistent upregulation of the cell death protein, Bik (FIG. 15), which presents a therapeutic opportunity to destroy them. Two major challenges facing cancer patients are therefore finding ways to predict whether tumors are prone to the dormant survival that promotes metastasis and identifying new therapies that will be effective against dormant cells. The present application teaches methods to both identify genetic markers to predict whether tumor cells will recur and exploit apoptotic pathway imbalance to kill dormant tumor cells.

FIG. 10 illustrates use of pathway analysis to identify p53 function and novel apoptotic regulators. To assess p53 function, two genes upregulated by the transcriptional activity of p53 (for example, Reprimo and GADD45) were used. These genes are expressed at low levels in wild-type cells and are induced with ionizing radiation via a p53-dependent mechanism. Other genes can be used. Without accounting for p53 function, the 50-60% of samples on a given microarray that have loss-of-function for p53 will confound the analysis of whether other apoptotic signaling proteins are altered.

FIG. 11 illustrates Bik protein sensitizes cells to apoptosis by counteracting Bcl-2 or Bcl-xL. Pro-survival Bcl-2 proteins (green), such as Bcl-2 and Bcl-xL promote cell survival by binding the pro-death proteins (red), such as Bax and Bak, which form pores in the outer mitochondrial membrane. Once pores of Bax and Bak form, cytochrome c is released from mitochondria and activates a cascade of caspase proteases that result in cell death. The Bik protein is an apoptotic “sensitizer” since it binds Bcl-2 or Bcl-xL and displaces apoptotic “activator” proteins, such as Bid and Bim, which are also sequestered by Bcl-2 or Bcl-xL. The apoptotic “activator” proteins promote pore formation by Bax and Bak, thereby directly activating cell death. Decreased levels of Bik (like those that we observed in the human breast tumor microarray dataset) would result in decreased sensitivity to apoptotic cell death. Apoptotic resistance through decreased Bik could therefore affect the metastatic capacity of tumor cells, by allowing them to survive in a rounded state during bloodborne transit.

FIG. 12 illustrates cell rounding also upregulates the BCRP drug resistance protein. BCRP is upregulated by cell rounding. MCF10A mammary epithelial cells or MCF-7 and Bt-474 breast tumor cells were grown for 24 h in standard media (Control), serum-free conditions (DMEM) or forced cell rounding through inhibition of actin polymerization with Latrunculin-A (LA, 5 μM—Rounded). Western blotting indicates that cell rounding increases BCRP protein in MCF10A and MCF-7 cells, but not Bt-474 cells. Levels of actin remain constant.

FIG. 13 illustrates cells expressing the survival proteins Bcl-2 or Bcl-xL are highly resistant to cell death induced by cell rounding (LA). (Left panel) When MCF10A cells are rounded with Latrunculin-A (24 h), cell viability drops significantly at doses as low as 2.5 μM. MCF10A cell lines that stably express either Bcl-2 (2.10) or Bcl-xL (2.12) are significantly resistant to apoptosis induced by cell rounding. Nearly 60% of cells overexpressing Bcl-xL remain viable even at concentrations of LA as high as 20 μM. (Right panel): Isolated analysis of the 5 μM dose shows that cells expressing Bcl-2 or Bcl-xL survive significantly more than parental MCF10A cells (P<0.05, t-test). Viability was measured with XTT and each point represents the mean+/−S.D. for triplicate samples.

FIG. 14 illustrates optical animal imaging shows that apoptotically-resistant cells survive dormantly. FIG. 14A shows stable expression of firefly luciferase in injected tumor cells allows imaging of tumor cell growth and spread in living mice by detecting the emitted firefly light with a sensitive CCD camera. When tumor cells are injected in the mammary gland to model primary breast cancer, light emission originates from mammary gland (right panel). FIG. 14B illustrates when tumor cells are injected into the bloodstream via the tail vein, they are trapped very quickly in the capillaries of the lung (as described in FIG. 1). Optical imaging shows the efficiency of this trapping of circulating tumor cells within 15 minutes of injection. FIG. 14C shows Mice that are injected with metastatic breast tumor cells (EpH4-MEK-Bcl2) (Martin et al., Oncogene, 23:4641-4645 (2004); and Pinkas et al., Mol. Cancer. Res., 2:551-556 (2004)), show early trapping in the lung and then a decrease in trapped cells over the first 48 hours as cells are pushed through the capillaries. After 20-30 days, tumor cells recur with the highest efficiency in the lung indicating that relatively fewer cells move successfully past the lung. The color scale here is logarithmic, and it is notable that the amount of tumor cells increases nearly 5000-fold during these 30 days. FIG. 14D shows tumor cells which resist apoptosis (BLuc cells express Bcl-2) follow a similar circulatory pattern, but do not begin to grow again, even after 20 days (right panels) and remain at less than 2-fold of the initial injection after 50 days (not shown). This technology allows us to accurately follow the spread of tumor cells via the circulation and measure whether tumor cells remain, even if they are in a dormant phase. Dormant tumor cells do not divide, but still produce light.

FIG. 15 illustrates apoptotically-resistant cell accumulate high levels of Bik when rounded. Western blotting shows that Bik levels are higher in MCF10A cells that stably express Bcl-2 or Bcl-xL when cells are grown normally, attached and spread on tissue culture dishes (Attached). Forced cell rounding with Latrunculin-A (LA, 5 μM−24 h) induces dramatic increases in Bik protein in cell lines expressing the survival proteins Bcl-2 or Bcl-xL. This increase in Bik is so strong that the moderate increases observed with LA in MCF10A cells (FIG. 7A) cannot even be detected here due to the short exposure times necessary to prevent oversaturating signal.

FIG. 16 illustrates apoptotic pathway imbalance may make resistant cells more sensitive to inhibitors of survival proteins than normal cells. The results of FIG. 15 suggest that mammary epithelial cells may compensate for high levels of prosurvival proteins by upregulating the Bik cell death protein. The high level of Bik in cells with elevated Bcl-2 may make these cells susceptible to inhibitors of Bcl-2 function. When Bcl-2 is inhibited with a small molecule compound like YC-137, the resulting imbalance in the Bcl-2/Bik ratio may result in cell death. Since MCF10A cells growing under normal conditions have such low levels of Bik (FIG. 15), inhibiting Bcl-2 may be far less toxic to these cells.

FIG. 17 illustrates the Bcl-2 inhibitor (YC-137) causes resistant 10A-Bcl2 cells to die when rounded. MCF10A cells stably expressing Bcl-2 (10A-Bcl2) are highly resistant to cell rounding and tolerate doses of LA as high as 20 μM (FIG. 13). Combining the moderate cell rounding effect of 2.5 μM LA with 1 μg/ml YC-137 led to a greater than 2-fold decrease in cell viability as gauged by XTT (bracket) compared to YC-137 alone. At doses higher than 1 μM, YC-137 apparently causes toxicity on its own, but at lower levels it affects rounded cells more than attached cells.

FIG. 18 illustrates the additive effect of YC137 and LA repeats, but dose and conditions must be optimized. In another study, YC-137 at either 1 μg/ml or 2.5 μg/ml synergized with LA-induced cell rounding (2.5 μM−24 h) to cause a significant decrease in cell viability (XTT assay) compared to cells treated with YC-137 alone (P<0.05, t-test).

FIG. 19 illustrates that YC137 remains toxic to normal cells (MCF10A). When MCF10A cells that do not express Bcl-2 are treated with YC137 at the indicated concentrations for 24 h, they also lose viability (XTT assay), and to a greater extent than 10A-Bcl2 cells. Combining YC-137 with cell rounding (LA, 2.5 μM−24 h) increases cell death in both MCF10A and 10A-Bcl2 cells. We tested a series of other Bcl-2 inhibitors (EM20-25, BH3I-1 and BH3I-2), but each of these display a similar nonspecific toxicity toward MCF10A cells. Such compounds would not be expected to be effective therapies due to likely side effects on normal tissues. If compounds were only toxic to rounded cells, that would be acceptable, since normal tissues are attached and not rounded. YC-137 is toxic to both attached and rounded MCF10A cells, indicating an unacceptable toxicity.

FIG. 20 illustrates that many existing Bcl-2 inhibitors are nonspecific, but a BH3-mimetic is specific. A recent publication shows that many of the current inhibitors of Bcl-2 function, including the BH3I-1″ that we have used, are toxic against cells that lack Bax and Bak (van Delft et al., Cancer Cell., 10(5):389-399 (2006)). FIG. 20A shows the viability of wild-type MEFs (WT) or Bax- and Bak-deficient MEFs (DKO) 24 hr after infection with the indicated retroviruses. Expression of the cDNA encoding the BH3-only protein BimS or tBid was linked by an IRES to that of GFP, and the viability of GFP+ cells was determined by PI exclusion. FIG. 20B illustrates Representative wells showing colony formation by wild-type (WT) or Bax/Bak-deficient (DKO) MEFs after infection with the control parental retrovirus or one expressing BimL. FIGS. 20C-20H show the viability (percent cells excluding PI) of WT or Bax- and Bak-deficient (DKO) MEFs treated for 24 hr with graded doses of the indicated putative BH3 mimetics. Since these Bcl-2 inhibitors (and YC137) are designed to mimic the BH3 domain that bind survival proteins Bcl-2 or Bcl-xL they should kill via a similar mechanism to the Bim or Bid BH3 domain retroviruses. Since these compounds continue to kill cells deficient in Bax or Bak, they clearly have significant nonspecific toxicity. FIG. 20F (right panel) shows that a BH3 mimetic (van Delft et al., Cancer Cell., 10(5):389-399 (2006)) causes decreases in cell viability that are progressively eliminated by knockout of either Bax or Bak and completely eliminated in the double knockout (DKO). This compound is an example of a BH3-mimetic for inhibiting Bcl-2 survival functions. BH3-mimetics are suitable for cell rounding and synergize with Bik elevation to specifically kill 10A-Bcl2 cells. BH3-mimetics or similar molecules are suitable to attain synergy.

FIG. 21 illustrates a two-pronged strategy to destroy circulating tumor cells. Both apoptotic pathway imbalance and microtentacles provide therapeutic opportunities that synergize to destroy circulating breast tumor cells and reduce metastatic recurrence. Inhibiting microtentacles prevents attachment and promote fragmentation of large epithelial carcinoma cells in narrow capillaries (Morris et al., Clin. Exp. Metastasis, 11:377-390 (1993); and Tsuji et al., Cancer Res., 66:303-306 (2006)). Cells escaping this biophysical destruction are susceptible to compounds targeting apoptotic imbalance, since they remain rounded in the circulation. We identify numerous targets for therapeutic compounds directed against microtentacles (tubulin carboxypeptidase, vimentin, kinesin) as well as those that would increase apoptotic pathway imbalance Inhibitors of Bcl-2 or Bcl-xL, for example, would allow accumulated Bik to cause cell death. Aromatase inhibitors offer an alternative method to downregulate Bcl-2 survival signals and increase Bax death signals (Thiantanawat et al., Cancer Res., 63:8037-8050 (2003)). Proteasome inhibitors would further increase Bik levels (FIG. 6). Simultaneously promoting fragmentation and apoptosis of circulating tumor cells outperforms either isolated therapy. Neither strategy requires active cell division, so each is a mechanistic alternative to therapies that target dividing cells. Surgery and other localized treatments increase circulating tumor cells (Momma et al., Cancer Res., 58:5425-5431 (1998); and Goldfarb et al., Breast Dis., 26:99-114 (2006)), so our approach aims to start the therapies before local treatment of the primary tumor (pre-adjuvant) and then continue treatment during and immediately after surgery. Targeting these two mechanisms helps reduce the survival of any tumor cells escaping the primary site during surgery or the following angiogenesis and wound healing.

FIG. 22 illustrates targeting Bik with siRNA to test its role in apoptosis. MCF10A cells were transfected with an siRNA directed against Bik mRNA (Qiagen: 5′-GAGGAGAAATGTCTGAAGTAA-3′ and incubated with the transfection complex for 3 days (72 hours). The media was then changed to either DMEM or DMEM with 5 μM LA for 16 hours. Bik protein levels were successfully down-regulated, as LA (5 μM, 24 h) was not able to induce Bik expression in the Bik siRNA-transfected cells. The DMEM media led to significant apoptosis.

FIG. 23 illustrates methods used to image circulating tumor cells and microtentacles. Surgically-isolated blood vessels from mice are sutured to the ends of glass micropipettes to allow solution flow through the vessels. After endothelial labeling with Fluo-4 (green), MDA-436 breast tumor cells, stained with QDot-655 (red), were injected and allowed to circulate through the vessel. An overlay image with DIC shows a tumor cell attached to the wall of a vessel with solution flowing through (FIG. 23 A). A side view of a confocal reconstruction (lower panel, right, FIG. 23B) shows the tumor cell penetrates the endothelium with microtentacle-like protrusions (arrows, FIG. 23 C). In the right panel the effects of microtentacle inhibitors are imaged with DIC microscopy. Detachment of vimentin-expressing breast tumor cell lines, like MDA-231 and MDA-436 (pictured, FIG. 23 D), stimulates the formation of rapidly-moving tubulin microtentacles (Whipple et al., “Vimentin filaments support extension of tubulin-based microtentacles in detached breast tumor cells”, Cancer Research, (2008), (under revision)). We added the kinesin inhibitor, tetracaine (100 μM) (Miyamoto et al., Biophys. J., 78:940-949 (2000)) and collected DIC images every 30 seconds (Yoon et al., “Microtentacles in detached breast tumor cells require kinesin motor activity”, manuscript in preparation (2008)). After approximately 10 minutes, tubulin microtentacles collapse dramatically (FIG. 23 E). This concentration of drug is non-toxic to these cells, even after 24 hours. Additional compounds targeting kinesins, tubulin detyrosination or vimentin assembly also inhibit microtentacles, but remain non-toxic.

### DETAILED DESCRIPTION OF THE INVENTION

### I. Definitions

As used herein, “a” or “an” may mean one or more. As used herein in the claims, when used in conjunction with the word “comprising”, the words “a” or “an” may mean one or more than one. As used herein “another” may mean at least a second or more. Furthermore, unless otherwise required by context, singular terms shall include pluralities and plural terms shall include the singular.

As used herein, “about” refers to numeric values whether or not explicitly indicated. The term “about” generally refers to a range of numbers (e.g., +/−5-10% of the recited value) that one would consider equivalent to the recited value (i.e., having the same function or result). In many instances, the term “about” may include numbers that are rounded to the nearest significant figure.

As used herein, a “sample” refers typically to any type of material of biological origin including, but not limited to, nucleic acids, proteins, lipids, an organelle, a cell, fluid, tissue, or an organ isolated from a subject, including, for example, DNA, RNA, mitochondria, nuclei, blood, plasma, serum, fecal matter, urine, semen, bone marrow, bile, spinal fluid, lymph fluid, samples of the skin, external secretions of the skin, respiratory, intestinal, and genitourinary tracts, tears, saliva, milk, blood cells, organs, or biopsies.

As used herein, “pathway” refers an interaction between more than one gene or gene products that depend on each other's individual function in order to make the aggregate function of the interaction of the more than one gene or gene product realized to the cell. Interactions between a gene or gene product includes, for example, phosphorylation, methylation, acylation, acetylation, alkylation, biotinylation, glycosylation, prenylation, sulfation, selenation, amidation, ISGylation, SUMOylation, ubiquitination, citrullination, deamidation, intra- and inter-disulfide bridge formation, ADP-ribosylation, binding to enable further interaction with other gene or gene products or assembly of a functional product, cleavage, addition or removal of a modifying group (e.g., addition or removal of lipids, sugars, amino acids, etc.), etc. Interactions also include, for example, any accompanying opposite function. For example the opposite function of phosphorylation is de-phosphorylation, the opposite function of methylation is de-methylation, the opposite function of acylation is de-acylation, and so forth.

As used herein, “cancer”, “cancer cells”, or “tumor” refers to, a pathophysiological state whereby a cell or cells are characterized by dysregulated and proliferative cellular growth and the ability to induce said growth, either by direct growth into adjacent tissue through invasion or by growth at distal sites through metastasis in both, adults or children, and both, acute or chronic, including, but not limited to, carcinomas and sarcomas, such as, acute lymphoblastic leukemia, acute myeloid leukemia, adrenocortical cancer, AIDS-related cancers, AIDS-related lymphoma, anal cancer, astrocytoma (cerebellar or cerebral), basal cell carcinoma, bile duct cancer, bladder cancer, bone cancer, brain stem glioma, brain tumor (e.g., ependymoma, medulloblastoma, supratentorial primitive neuroectodermal, visual pathway and hypothalamic glioma), cerebral astrocytoma/malignant glioma, breast cancer, bronchial adenomas/carcinoids, Burkitt's lymphoma, carcinoid tumor (e.g., gastrointestinal), carcinoma of unknown primary site, central nervous system lymphoma, cervical cancer, chronic lymphocytic leukemia, chronic myelogenous leukemia, chronic myeloproliferative disorders, colon cancer, colorectal cancer, cutaneous T-Cell lymphoma, endometrial cancer, ependymoma, esophageal cancer, Ewing's Family of tumors, extrahepatic bile duct cancer, eye cancer (e.g., intraocular melanoma, retinoblastoma, gallbladder cancer, gastric cancer, gastrointestinal carcinoid tumor, gastrointestinal stromal tumor (GIST), germ cell tumor (e.g., extracranial, extragonadal, ovarian), gestational trophoblastic tumor, glioma, hairy cell leukemia, head and neck cancer, squamous cell head and neck cancer, hepatocellular cancer, Hodgkin's lymphoma, hypopharyngeal cancer, islet cell carcinoma (e.g., endocrine pancreas), Kaposi's sarcoma, laryngeal cancer, leukemia, lip and oral cavity cancer, liver cancer, lung cancer (e.g., non-small cell), lymphoma, macroglobulinemia, malignant fibrous histiocytoma of bone/osteosarcoma, medulloblastoma, melanoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myeloma, nasal cavity and paranasal sinus cancer, nasopharyngeal cancer, neuroblastoma, non-Hodgkin's lymphoma, oral cancer, oral cavity cancer, osteosarcoma, oropharyngeal cancer, ovarian cancer (e.g., ovarian epithelial cancer, germ cell tumor), ovarian low malignant potential tumor, pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, prostate cancer, rectal cancer, retinoblastoma, rhabdomyosarcoma, salivary gland cancer, soft tissue sarcoma, uterine sarcoma, Sézary syndrome, skin cancer (e.g., non-melanoma or melanoma), small intestine cancer, supratentorial primitive neuroectodermal tumors, T-Cell Lymphoma, testicular cancer, throat Cancer, thymoma, thymoma and thymic carcinoma, thyroid Cancer, transitional cell cancer of the renal pelvis and ureter, trophoblastic tumor (e.g., gestational), unusual cancers of childhood and adulthood, urethral cancer, endometrial uterine cancer, uterine sarcoma, vaginal cancer, vulvar cancer, Waldenström's macroglobulinemia, Wilms' Tumor, and women's cancers.

As used herein, “microtentacles”, refers to, dynamic protrusions of detached tumor cells, including breast tumor cells. (Whipple et al., Exp. Cell Res., 313(7):1326-36. (2007)).

As used herein, “apoptotic pathway imbalance”, refers to, detached cells that avoid apoptosis and survive dormantly while continuing to accumulate pro-apoptotic proteins, such as Bik.

As used herein, “apoptosis inducing” and “pro-apoptotic” refers at least to apoptosis promotion, induction, and enhancement.

As used herein, “anti-apoptotic” refers at least to apoptosis blockade, neutralization, suspension, diminishment and decrease.

As used herein, “anti-apoptotic agent” refers to anything capable of protecting a host cell containing the agent against apoptosis. The anti-apoptotic agent utilized in the invention can be selected from the anti-apoptotic members of the Bcl-2 family of genes. For example, the ability of Bcl2 to protect against anti-Fas antibody-induced liver injury is known in the art (i.e., V. Lacronique et al., Nature Med., 2(1):80-86 (January 1996). The cDNA sequence of Bcl2 is known in the art (i.e., Y. Tsujimoto & C. M. Croce, Proc. Natl. Acad. Sci. USA, 83:5214-5218 (1986)). An ordinarily skilled artisan recognizes that other anti-apoptotic members of the Bcl-2 family can be routinely substituted. Alternatively, other inhibitors of interleukin-1beta-converting enzyme (ICE)-type proteases and/or inhibitors of apoptosis may be substituted for Bcl-2, and the apoptotic agent utilized in the invention adjusted accordingly using art known methods. Reference to Bcl-2 is exemplary. Other anti-apoptotic agents may be readily utilized in the method and constructs of the invention.

### II. The Present Invention

Apoptosis, or programmed cell death is important for normal development, host defense and suppression of oncogenesis. Faulty regulation of apoptosis has been implicated in cancer and many other human diseases. Bcl-2 was originally identified at the chromosomal breakpoint of t(14; 18)-bearing B-cell lymphomas and belongs to a growing family of proteins which regulates apoptosis. (Reed, J. Cell. Biol., 124:1-6 (1994); Reed, Nature, 387:773-776 (1997); Hawkins et al., Immunological Reviews, 142:127-139 (1994); and Minn et al., Advances in Immunology, 70:245-279 (1998)). In cancerous B cells, the portion of chromosome 18 containing the bcl-2 locus undergoes a reciprocal translocation with the portion of chromosome 14 containing the antibody heavy chains. This t(14; 18) translocation places the bcl-2 gene close to the heavy chain gene enhancer. The product of the Bcl-2 gene, Bcl-2 protein, is an integral membrane protein found in the membranes of the endoplasmic reticulum (ER), nuclear envelope, and the outer membrane of mitochondria.

The Bcl-2 family of proteins includes both anti-apoptotic molecules, for example, Bcl-2 and Bcl-xL and pro-apoptotic molecules, for example, Bax, Bak, Bid, Bik, and Bad. These molecules play an important role in regulating apoptosis. (Chao et al., Annul. Rev. Immunol., 16:395-419 (1998); Gross et al., Genes & Develop., 13:899-1911 (1999); Hawkins et al., Semin. Immunol., 9:25-33 (1997); Reed, Oncogene, 18:3225-3236 (1998); Park et al., J. Cell. Biochem., 60:12-17 (1996); Reed, J. Cell. Biol., 124:1-6 (1994); Reed, Nature, 387:773-776 (1997); Reed et al., J. Cell. Biochem., 60:23-32 (1996); Adams et al., Science, 281:1322-1326 (1998); Hawkins et al., Immunol. Rev., 142:127-139 (1994)).

In the present specification the potential of the Bcl-2 family of proteins as pharmaceutically or diagnostically active substances in cancer has been studied with particular reference to the Bcl-2 protein. In addition, the potential of Bcl-X_{L} and Mcl-1 as pharmaceutically and diagnostically active substance is known in the art. Immune responses like those elicited against the Bcl-2 protein or fragments can be observed in cancer patients against other members of the Bcl-2 protein family, e.g. other anti-apoptotic proteins such as Mcl-1 or Bcl-X_{L}, which are also related to drug resistance and over-expression in cancer. Accordingly, the invention pertains to any member of the Bcl-2 protein family, or any apoptotic signaling protein or mechanism. It is known in the art that the Bcl-2 anti-apoptotic family members exert oncogenic effects via inhibition of apoptosis in cells that are scheduled to die, thereby resulting in an accumulation of cells. Members of the Bcl-2 protein family contain at least one of four conserved motifs known as Bcl-2 homology (BH) domains (BH1, BH2, BH3, and BH4). In addition to the presence of BH domains, preferred anti-apoptotic molecules possess a carboxyl-terminal membrane-anchoring domain (TM). It is known in the art that anti-apoptotic members such as Bcl-2 and Bcl-X_{L} contain all four BH domains, along with the transmembrane domain. Multidomain pro-apoptotic proteins such as Bax and Bak contain all but the BH4 domain. A second subgroup of pro-apoptotic proteins, known as BH3-domain only proteins (including Bad and Bid), consists of molecules that contain only the BH3 domain and lack other BH domains. Proapoptotic proteins such as Bcl-X_{S} and Mcl-1_{S}, representing alternatively spliced forms of the bcl-x and mcl-1 genes, respectively, lack BH1 and BH2 domains. Additionally, Mcl-1_{S} lacks a transmembrane domain. Proteins belonging to the Bcl-2 family are known in the art. Even though it is preferred that the protein belonging to the Bcl-2 protein family has anti-apoptotic properties, it is also within the present invention that the protein belonging to the Bcl-2 family may be a pro-apoptotic protein, for example a protein selected from the group consisting of Bax, Bok/Mtd, Bad, Bik/Nbk, Bid, Hrk/DP5, Bim, Noxa, Bmf and PUMA/bbc3. Survivin and exemplary diagnostic and therapeutic uses of survivin are known in the art (i.e., U.S. Pat. No. 6,245,523, herein incorporated by reference). Survivin is a 16.5 kDa cytoplasmic protein containing a single BIR and a charged carboxy-terminal coiled coil region instead of a RING finger, which inhibits apoptosis induced by growth factor (IL-3) withdrawal when transferred in B cell precursors. The gene coding for survivin is nearly identical to the sequence of Effector Cell Protease Receptor-1 (EPR-1) however, is oriented in the opposite direction. Thus, survivin is considered an antisense EPR-1 product in the art Inhibition of survivin by increases in natural antisense EPR-1 transcript results in apoptosis and cell cycle arrest. U.S. Pat. No. 6,245,523 indicates isolation of purified surviving, provides nucleic acid molecules that encode survivin, antibodies, other molecules that bind to surviving, anti-apoptotically active fragments of survivin wherein an amino acid residue is inserted N- or C-terminal to, or within, survivin. Peptides containing functional residues required for apoptosis, i.e. Trp at position 67, Pro at position 73 and Cys at position 84 are indicated.

Although an understanding of the mechanism is not necessary to practice the present invention and the present invention is not so limited, it is contemplated that anti-apoptotic proteins, such as Bcl-2 and Bcl-xL suppress apoptosis by forming heterodimers with pro-apoptotic Bcl-2 family members such as Bak, Bad, Bid, Bax, Mtd (Bok), Bim, Hrk (DP5), Blk, Bik, Bnip3, Bnip3L, Bmf, Noxa, Puma, Nix, and Diva. Additional anti-apoptotic members (or related proteins) of the Bcl-2 family include, but are not limited to, Mcl-1, A-1 (Bfl-1), Boo, NR-13, and Bcl-W.

The invention is based, in part, on exploiting imbalances in apoptotic pathways in cancer cells for the treatment of cancer. A mechanism by which cancer cells are able to grow, and continue to grow, is the overexpression of antiapoptotic genes, such as, Bcl-2 family members, Bcl-2, Bcl-xL, or both, or similar proteins or combinations thereof, the reduced expression of proapoptotic genes. The invention is also based on methods of cancer prognosis and screening for therapeutic targets for cancer by analyzing gene expression wherein the analysis is carried out in samples that have been determined to have positive p53 function.

Our lab has identified two novel characteristics of detached breast tumor cells that provide therapeutic opportunities to destroy circulating tumor cells, independent of cell division. The first is our discovery that detached breast tumor cells generate dynamic protrusions, that we have termed tubulin microtentacles (Whipple et al., Exp. Cell Res., 313:1326-1336 (2007). A role for these novel microtentacles in metastasis is supported by compelling in vivo evidence that a yet-unidentified, tubulin-based (and not actin-based) mechanism is required for circulating tumor cells to engage blood vessel walls (Korb et al., Exp. Cell Res., 299:236-247 (2004)). Second, detached cells that avoid apoptosis can survive dormantly (Martin et al., Oncogene, 23:4641-4645 (2004); and Pinkas et al., Mol. Cancer Res., 2:551-556 (2004)), but continue to accumulate pro-apoptotic protein, such as Bik protein, a process we call apoptotic pathway imbalance. This persistent upregulation of, for example, Bik renders circulating tumor cells highly susceptible to inhibition of survival pathways. Since rounded epithelial cells have exceptionally high levels of Bik, it is possible to promote tumor cell death and spare normal tissues by exploiting this imbalance in apoptotic signaling. Microtentacles are discussed in P03281WO0, herein incorporated by reference in its entirety, while apoptotic pathway imbalance is discussed in PCT US07/063,566, herein incorporated by reference in its entirety. Thus, therapies targeting microtentacles and apoptotic pathway imbalance synergize to destroy circulating tumor cells.

### Methods of Treating Cancer

In particular embodiments, the invention is drawn to a method of treating cancer in a subject in need of such treatment comprising the administration to said subject of an effective dose of an apoptosis inducing agent selected from the group consisting of a member of the Bcl-2 family of proteins, or combinations thereof, for example, a Bcl-2 inhibitor and a Bcl-xL inhibitor. A subject treated using the methods of the invention encompasses a recipient of the method practiced. The subject can be any unicellular or multicellular organism, including a mammal. A mammal includes, but is not limited to, human beings, domesticated animals (such as, for example, dogs, cats, and hamsters), and livestock (such as, for example, cows, pigs, sheep, and chickens). An effective dose of an apoptosis inducing agent is a dose of an agent that affects apoptosis in a cell or cells and is determined by one of ordinary skill in the art. Affecting apoptosis can be either increasing or maintaining apoptosis. The precise determination of the effective dose is accomplished by one of ordinary skill in the art that administers an apoptosis inducing agent.

In particular embodiments, the invention is drawn to a method of killing a cancer cell comprising contacting said cancer cell with an effective dose of an apoptosis inducing agent selected from the group consisting of a Bcl-2 inhibitor and a Bcl-xL inhibitor. In other embodiments, contacting the cancer cell with an effective dose of an apoptosis inducing agent is carried out in combination with one or more anticancer agents or radiation therapy.

In particular embodiments, apoptosis inducing agents of the invention include, but are not limited to, Bcl-2 and Bcl-xL inhibitors. Bcl-2 inhibitors promote apoptosis since Bcl-2 is antiapoptotic. Bcl-2 inhibitors include any molecular entity that inhibits or interferences with Bcl-2 activity, including, but not limited to, a small molecule, nucleic acid (such as, for example, siRNA, shRNA expression cassette, antisense DNA, and antisense RNA), protein, peptide, antibody, antibody fragment, antisense drug, or other biomolecule that is naturally made, synthetically made, or semi-synthetically made. Based on the state of the art one of ordinary skill in the art understands how to obtain compounds, for example as demonstrated by [insert patent references to each type of compound as of provisional filing date]. Like Bcl-2, Bcl-xL inhibitors promote apoptosis since Bcl-xL is also antiapoptotic. Bcl-xL inhibitors include any molecular entity that inhibits or interferes with Bcl-xL activity, including, but not limited to, a small molecule, nucleic acid (such as, siRNA, shRNA expression cassette, antisense DNA, antisense RNA, etc.), protein, peptide, antibody, antibody fragment, antisense drug, or other biomolecule that is naturally made, synthetically made, or semi-synthetically made. The invention is drawn, in part, to exploiting imbalances in the apoptotic pathway and is thus not limited by any particular means of exploiting this pathway. Bcl-2 and Bcl-xL inhibitors can act directly on their respective biomolecule or can also act indirectly on their respective biomolecule. For example, inhibitors can act on upstream or downstream regulators of Bcl-2 or Bcl-xL such that the antiapoptotic activities of Bcl-2 or Bcl-xL are not realized by the cell or cells.

Additional anti-apoptotic members (or related proteins) of the Bcl-2 family include, but are not limited to, Mcl-1, A-1 (Bfl-1), Boo, NR-13, and Bcl-W. Apoptosis inducing agents of the invention include inhibitors of these molecules and other anti-apoptotic molecules. Apoptosis inducing agents directed at these molecules include inhibitors including any molecular entity that inhibits or interferences with their respective anti-apoptotic activity, including, but not limited to, a small molecule, nucleic acid (such as, for example, siRNA, shRNA expression cassette, antisense DNA, and antisense RNA), protein, peptide, antibody, antibody fragment, antisense drug, or other biomolecule that is naturally made, synthetically made, or semi-synthetically made. The invention is drawn, in part, to exploiting imbalances in the apoptotic pathway and is thus not limited by any particular means of exploiting this pathway. The inhibitors can act directly or indirectly to decrease anti-apoptotic activity. For example, inhibitors can act on upstream or downstream regulators of Mcl-1, A-1 (Bfl-1), Boo, NR13, or Bcl-W such that the antiapoptotic activities of Mcl-1, A-1 (Bfl-1), Boo, NR13, or Bcl-W are not realized by the cell or cells.

In other embodiments, the invention drawn to apoptosis inducing agents includes pro-apoptotic molecules, such as, for example, as Bak, Bad, Bid, Bax, Mtd (Bok), Bim, Hrk (DP5), Blk, Bik, Bnip3, Bnip3L, Diva, Noxa, Puma, Bmf, Nix and molecules that increase the activity of these pro-apoptotic molecules or other pro-apoptotic molecules. Apoptosis inducing agents directed at these molecules includes any molecular entity that increases or maintains their respective pro-apoptotic activity, including, but not limited to, a small molecule, nucleic acid (such as, for example, siRNA, shRNA expression cassette, antisense DNA, and antisense RNA), protein, peptide, antibody, antibody fragment, antisense drug, or other biomolecule that is naturally made, synthetically made, or semi-synthetically made. The invention is drawn, in part, to exploiting imbalances in the apoptotic pathway and is thus not limited by any particular means of exploiting this pathway. The pro-apoptotic molecules can act directly or indirectly to increase pro-apoptotic activity. For example, pro-apoptotic molecules can act on upstream or downstream regulators of Bak, Bad, Bid, Bax, Mtd (Bok), Bim, Hrk (DP5), Blk, Bik, Bnip3, Bnip3L, Bmf, Noxa, Puma, Nix or Diva such that the pro-apoptotic activities of Bak, Bad, Bid, Bax, Mtd (Bok), Bim, Hrk (DP5), Blk, Bik, Bnip3, Bnip3L, Bmf, Noxa, Puma, Nix or Diva are realized by the cell or cells.

In addition to a single therapy, which is the case, for example, with the administration of a single Bcl-2 or Bcl-xL inhibitor, cancer treatments are commonly combined with other methods of treating cancer. Combination therapy includes combining the method of treating cancer as described in the invention and one or more cancer therapeutic methods. Cancer therapeutic methods include surgical therapy, radiation therapy, administering an anticancer agent (including, for example, antineoplastics and angiogenesis inhibitors), immunotherapy, antineoplastons, investigational drugs, vaccines, less conventional therapies (sometimes referred to as novel or innovative therapies, which include, for example, chemoembolization, hormone therapy, local hyperthermia, photodynamic therapy, radiofrequency ablation, stem cell transplantation, and gene therapy), prophylactic therapy (including, for example, prophylactic mastectomy), and alternative and complementary therapies (including, for example, dietary supplements, megadose vitamins, herbal preparations, special teas, physical therapy, acupuncture, massage therapy, magnet therapy, spiritual healing, meditation, pain management therapy, and naturopathic therapy (including, for example, botanical medicine, homeopathy, Chinese medicine, and hydrotherapy).

In particular embodiments, the invention for treating cancer (e.g., inhibitors of a member of the Bcl-2 family or combinations thereof, for example Bcl-2 or Bcl-xL inhibitors) can be used alone, in combination with an anticancer agent, or in combination with an anticancer combination (i.e., a combination of anticancer agents).

In other embodiments, an anticancer agent can be, for example, Abraxane, Aldara, Alimta, Aminolevulinic Acid, Anastrozole, Aprepitant, Arimidex, Aromasin, Arranon, Arsenic Trioxide, Avastin, Azacitidine, Bevacizumab, Bexarotene, Bortezomib, Capecitabine, Cetuximab, Cisplatin, Clofarabine, Clofarex, Clolar, Dacogen, Dasatinib, Decitabine, Docetaxel, Ellence, Eloxatin, Emend, Epirubicin Hydrochloride, Erbitux, Erlotinib, Exemestane, Faslodex, Femara, Fulvestrant, Gefitinib, Gemcitabine, Gemtuzumab Ozogamicin, Gemzar, Gleevec, Herceptin, Hycamtin, Imatinib Mesylate, Imiquimod, Iressa, Kepivance, Lenalidomide, Letrozole, Levulan, Methazolastone, Mylosar, Mylotarg, Nanoparticle Paclitaxel, Nelarabine, Nexavar, Nolvadex, Oncaspar, Oxaliplatin, Paclitaxel, Paclitaxel Albumin-stabilized Nanoparticle Formulation, Palifermin, Panitumumab, Pegaspargase, Pemetrexed Disodium, Platinol-AQ, Platinol, Revlimid, Rituxan, Sclerosol Intrapleural Aerosol, Sorafenib Tosylate, Sprycel, Sterile Talc Powder, Sunitinib Malate, Sutent, Synovir, Tamoxifen, Tarceva, Targretin, Taxol, Taxotere, Temodar, Temozolomide, Thalomid, Thalidomide, Topotecan Hydrochloride, Trastuzumab, Trisenox, Vectibix, Velcade, Vidaza, Vorinostat, Xeloda, Zoledronic Acid, Zolinza, Zometa, doxorubicin, adriamycin, bleomycin, daunorubicin, dactinomycin, epirubicin, idarubicin, mitoxantrone, valrubicin, hydroxyurea, mitomycin, fluorouracil, 5-FU, methotrexate, floxuridine, interferon alpha-2b, glutamic acid, plicamycin, 6-thioguanine, aminopterin, pemetrexed, raltitrexed, cladribine, clofarabine, fludarabine, mercaptopurine, pentostatin, capecitabine, cytarabine, carmustine, BCNU, lomustine, CCNU, cytosine arabinoside, cyclophosphamide, estramustine, hydroxyurea, procarbazine, mitomycin, busulfan, medroxyprogesterone, estramustine phosphate sodium, ethinyl estradiol, estradiol, megestrol acetate, methyltestosterone, diethylstilbestrol diphosphate, chlorotrianisene, testolactone, mephalen, mechlorethamine, chlorambucil, chlormethine, ifosfamide, bethamethasone sodium phosphate, dicarbazine, asparaginase, mitotane, vincristine, vinblastine, etoposide, teniposide, Topotecan, IFN-gamma, irinotecan, campto, irinotecan analogs, carmustine, fotemustine, lomustine, streptozocin, carboplatin, oxaliplatin, BBR3464, busulfan, dacarbazine, mechlorethamine, procarbazine, thioTEPA, uramustine, vindesine, vinorelbine, alemtuzumab, tositumomab, methyl aminolevulinate, porfimer, verteporfin, lapatinib, nilotinib, vandetanib, ZD6474, alitretinoin, altretamine, amsacrine, anagrelide, denileukin diftitox, estramustine, hydroxycarbamide, masoprocol, mitotane, tretinoin, or other anticancer agents, including, for example, antibiotic derivatives, cytotoxic agents, angiogenesis inhibitors, hormones or hormone derivatives, nitrogen mustards and derivatives, steroids and combinations, and antimetholites. In further particular embodiments, an anticancer agent comprises two or more of the foregoing anticancer agents.

In other embodiments an anticancer combination includes, for example, CHOP (Cytoxan, Hydroxyrubicin (Adriamycin), Oncovin (Vincristine), Prednisone), CHOP-R (CHOP, rituximab), FOLFOX (Fluorouracil, leucovorin (folinic acid), oxaliplatin), VAD (Vincristine, Adriamycin (doxorubicin), dexamethasone), Thal/Dex (Thalidomide, dexamethasone), COP or CVP (Cyclophosphamide, vincristine (Oncovin), and prednisone), m-BACOD (Methotrexate, bleomycin, doxorubicin (Adriamycin), cyclophosphamide, vincristine (Oncovin), dexamethasone (Decadron)), ProMACE-CytaBOM (Prednisone, doxorubicin (adriamycin), cyclophosphamide, etoposide, cytarabine, bleomycin, vincristine (Oncovin), methotrexate, leucovorin), COPP (Cyclophosphamide, Oncovin (vincristine), procarbazine, prednisone), MACOP-B (Methotrexate, leucovorin, doxorubicin (Adriamycin), cyclophosphamide, vincristine (Oncovin), prednisone, bleomycin), MOPP (Mechlorethamine, vincristine (oncovin), procarbazine, prednisone), ProMACE-MOPP (Methotrexate, doxorubicin (Adriamycin), cyclophosphamide, etoposide, MOPP), ABVD (Adriamycin, bleomycin, vinblastine, dacarbazine), BEACOPP (Bleomycin, etoposide, Adriamycin (doxorubicin), cyclophosphamide, Oncovin (vincristine), procarbazine, prednisone), Stanford V (Doxorubicin (Adriamycin), mechlorethamine, bleomycin, vinblastine, vincristine (Oncovin), etoposide (VP-16), prednisone), and ECF (Epirubicin, cisplatin, fluorouracil), BEP (Bleomycin, etoposide, platinum (cisplatin)), PCV (Procarbazine, lomustine (CCNU), vincristine).

In particular embodiments, the invention for treating cancer (i.e., inhibitors of a member of the Bcl-2 family or combinations thereof, for example Bcl-2 or Bcl-xL inhibitors) can be used alone or in combination with radiation therapy (also called radiotherapy, x-ray therapy, irradiation, etc.). Inhibitors of a member of the Bcl-2 family or combinations thereof, for example Bcl-2 or Bcl-xL inhibitors can be administered prior to, concomitant with, or after radiation therapy. Radiation therapy is the use of certain types of high-energy radiant to kill cancer cells and shrink tumors or as prophylactic treatment to prevent cancer. Generally, radiation therapy uses high-energy radiation from, for example, x-rays, gamma rays, neutrons, and other sources. Radiation may be external in origin (e.g., come from a machine outside the body, external-beam radiation therapy), or may originate from radioactive material placed in the body (e.g., internal radiation therapy, implant radiation, or brachytherapy). Systemic radiation therapy uses a radioactive substance (e.g., radiopharmaceuticals, radioactive drugs, radionucleotides, etc.) such as a radiolabeled monoclonal antibody directed to cancer cells, that circulates throughout the body. Types of radiation therapy include, but are not limited to, intraoperative radiation therapy, prophylactic cranial irradiation, interstitial radiation, intracavitary or intraluminal radiation, stereotactic radiation, 3-D conformal radiation, external beam radiation, high-dose rate (HDR) brachytherapy, intensity modulated radiation therapy (IMRT), MammoSite radiation therapy system (RTS), TheraSphere, TomoTherapy highly integrated adaptive radiotherapy (HI-ART), etc. Radiation therapy can also be used in combination with radiosensitizers and radioprotectors, which are entities that modify a cell's response to radiation. Radiosensitizers make cells more sensitive to the effects of radiation whereas radioprotectors make cells less sensitive to the effects of radiation. Several compounds are under study as radiosensitizers. In addition, some anticancer drugs, such as, for example, 5-fluorouracil and cisplatin, make cancer cells more sensitive to radiation therapy. Hyperthermia, the use of heat, can also be used in conjunction with radiation therapy. The combination of heat and radiation can increase the response rate of some tumors.

In particular embodiments, the invention for treating cancer (i.e., inhibitors of a member of the Bcl-2 family or combinations thereof, for example Bcl-2 or Bcl-xL inhibitors) can be used alone or in combination with surgical therapy. At least one inhibitor of a member of the Bcl-2 family or combinations of inhibitors, for example Bcl-2 or Bcl-xL inhibitor can be administered prior to, concomitant with, or after surgical therapy. In specific embodiments, inhibitors of a member of the Bcl-2 family or combinations thereof, for example Bcl-2 or Bcl-xL inhibitor is administered prior surgical therapy. In further specific embodiments, a Bcl-2 or Bcl-xL inhibitor is administered prior to surgical therapy so as to prevent metastasis of cancer cells undergoing surgical therapy. Generally, surgical therapy is an invasive cancer therapy whereby physical removal of cancer cells is the objective. Surgical therapy includes, for example, tumor resection and cavitron ultrasonic surgical aspiration (CUSA).

In particular embodiments, the invention is drawn to a method of preventing tumor metastasis in a subject in need of such prevention comprising the administration to said subject of an apoptosis inducing agent selected from the group consisting of at least one inhibitor of a member of the Bcl-2 family or combinations thereof, for example Bcl-2 inhibitor and a Bcl-xL inhibitor. In other embodiments, the apoptosis inducing agent of the invention is administered in combination with one or more methods of treating cancer selected from the group consisting of chemotherapy, radiation therapy, and surgical therapy. In specific embodiments, the apoptosis inducing agent is administered in combination with surgical therapy. In other specific embodiments, the apoptosis inducing agent is administered in advance of surgical therapy. In other specific embodiments, the surgical therapy is surgical tumor resection.

Metastasis is the spread of cancer cells from one part of the body to another. A tumor formed by cells that have spread is called a “metastatic tumor” or a “metastasis.” Post-surgery metastasis is clinically observed. For example, surgical removal of cancer cells can cause a release of cancer cells into the blood stream resulting in the transplantation of cancer cells at some site in the body that is different from an original tumor site (Leondi et al., International Seminars in Surgical Oncology, 2:7 (2005)). Additionally, other mechanisms can be involved in the transplantation of cancer cells to a particular site or sites in the body that is different from an original tumor site. For example, detachment of primary tumor cells into the bloodstream, lymphatic vessels or interorgan spaces is required for tumor metastasis. This detachment causes cell rounding, a stimulus that induces apoptotic cell death in adherent cell types, such as those that compose solid tumors. Tumor cells that resist apoptosis can survive the long-term cell rounding that occurs during detachment, an attribute that promotes the colonization of distant sites. Eventual outgrowth of the tumor can be delayed for months or years, as apoptotic resistance is not sufficient for immediate tumor regrowth and promotes dormant survival of tumor cells (Martin et al., Oncogene, 23:4641-4645 (2004); and Pinkas et al., Molecular Cancer Research, 2:551-556 (2004)). Since these cells survive without active cell division, they are highly resistant to classical chemotherapies which target dividing cells (Naumov et al., Breast Cancer Res. Treat., 82:199-206 (2003)). Apoptotically-resistant tumor cells may therefore serve as a reservoir in distant tissues for the eventual recurrence of metastatic tumors. Destroying these apoptotically-resistant cells could help avoid tumor recurrence, even years later.

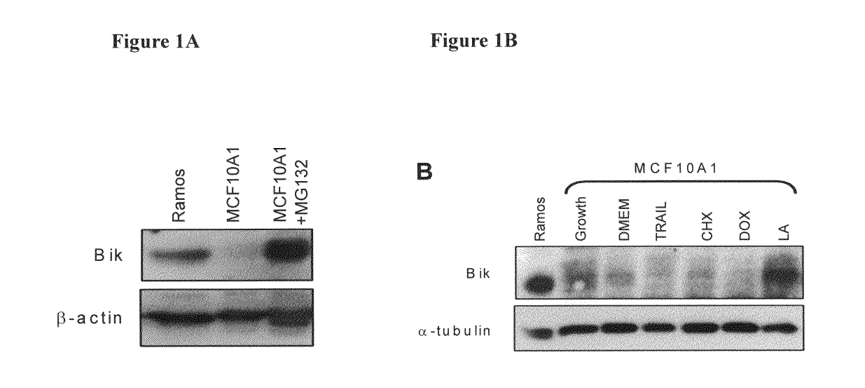

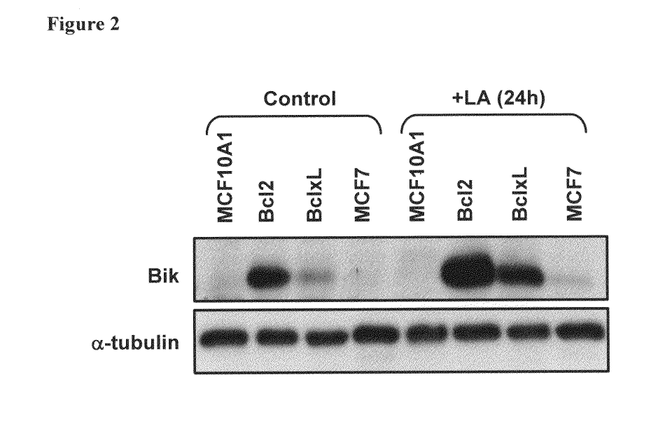

Apoptotic resistance can be mediated by alterations and/or the ratio of apoptotic mediators. Without being bound by theory, it is believed that apoptotic mediators, such as, for example, Bik, are upregulated in these cells, but although there are high levels of these apoptotic mediators, prosurvival or antiapoptotic mediators, such as, for example, Bcl-2 and/or Bcl-xL are also elevated in these cells, contributing increased resistance to apoptosis in cells undergoing rounding (FIGS. 1 and 2). An elevation of Bik, or like apoptotic mediators, may make these cells more sensitive to a moderate inhibition of Bcl-2 or Bcl-xL, or like prosurvival or antiapoptotic mediators. Since normal cells would not have such high levels of Bik, or like apoptotic mediators, they might remain unaffected by low levels of Bcl-2 or Bcl-xL inhibition, or inhibition of like prosurvival or antiapoptotic mediators. Under such a situation, a critical point to destroy tumor cells may be during their circulation in the bloodstream. Surgery or treatment of the primary tumor increases the levels of circulating tumor cells in the bloodstream, likely either as a result of the procedure itself or the wound healing that follows (Momma et al., Cancer Res., 58:5425-5431, PMID: 9850075 (1998)). For this reason, pretreating patients with an apoptosis inducing agent, such as, for example, inhibitors of a member of the Bcl-2 family or combinations thereof, for example Bcl-2 or Bcl-xL inhibitor, or inhibitors of like prosurvival or antiapoptotic mediators, immediately before surgery and/or for a short period after surgery may improve the destruction of tumor cells that escape the primary site during surgery and the wound healing that follows.

### Methods of Cancer Diagnosis, Prognosis, and Screening for Cancer Therapy Targets

Loss of function of certain genes is common in cancer, for example p53 (Royds, Cell Death Differ., 13:1017-1026 (2006)). There are numerous posttranscriptional mechanisms that regulate gene activity so gene expression data by itself is not adequate to determine gene and pathway function. A novel and more effective approach is to identify transcriptional targets as indicators of function and allow filtration of gene expression data and any indicators of diagnosis, prognosis, or therapeutic targets for cancer on the basis of intact gene and pathway function.

In particular embodiments, the invention is drawn to a method of analyzing a sample utilizing gene expression data related to cancer using a filter mechanism whereby said mechanism comprises using a first gene as an indicator of the function of a second gene, for example, a gene related to cancer, allowing for distinguishing said sample as functional or non-functional (i.e., loss of function) for the second gene and a functional pathway associated therewith. In other embodiments, the invention is drawn to analyzing the distinguished samples to determine prognostic indicators related to cancer. However, the method described herein is also modified so as to serve as method of determining, for example, diagnostic indicators or therapeutic targets for cancer. For example, the invention is used to determine overexpression or underexpression of a gene in relation to the expression level when compared to a control, which serves as a diagnostic indicator of cancer or serves as a therapeutic target for cancer.

In specific embodiments, the first gene serving as an indicator of the function of a second gene is Reprimo, GADD45, or both Reprimo and GADD45, or similar suitable proteins or combinations thereof. In other specific embodiments, the second gene for which Reprimo, GADD45, or both Reprimo and GADD45 indicate function is p53 and a functional pathway associated therewith. The first gene serving as an indicator of function is not limited to Reprimo and/or GADD45. The first gene serving as an indicator is only limited by its ability to act as an indicator of a second gene and a functional associated pathway. Suitable first genes are known in the art. For example, a first gene serving as an indicator may be regulated by a second gene or pathway associated therewith such that the function of the second gene or pathway associated therewith results in maintained or elevated expression of the first gene serving as an indicator. Alternatively, a first gene serving as an indicator may be regulated by a second gene or pathway associated therewith such that the loss of function of the second gene or pathway associated therewith results in decreased expression of the first gene serving as an indicator. The function of a first gene serving as an indicator is that it is associated in some manner (e.g., decreased, maintained, or increased expression) with the function and/or loss of function of a second gene and a functional pathway associated therewith. Similarly as to a first gene serving as an indicator, a second gene or pathway associated therewith is not limited to p53. Suitable second genes are known in the art. Any gene or pathway associated therewith is within the scope of the invention so long as said gene or pathway associated therewith is used to filter gene expression data and indicate a diagnostic, prognostic, or therapeutic target for cancer. The gene and pathway function can be intact (i.e., functional) or loss of function.

In particular embodiments, the invention as described herein using a filter mechanism by which to analyze gene expression data is drawn to determining a pharmacogenetic marker. A “pharmacogenetic marker” is an objective biochemical marker that correlates with a specific clinical drug response or adverse reaction (see, for example, Mcleod et al., Eur. J. Cancer, 35:1650-1652 (1999). The presence or quantity of the pharmacogenetic marker is related to the predicted response of the subject to a specific drug or class of drugs prior to administration of the drug. By assessing the presence or quantity of one or more pharmacogenetic markers in a subject, a drug therapy (e.g., a specific drug or drugs, dosing, or dosing regimen) that is most appropriate for the subject or that is predicted to have a greater degree of success can be selected.

While the invention has been described with reference to specified embodiments thereof, those skilled in the art will appreciate that various modifications may be made without departing from the spirit and scope of the invention. The invention is not limited in any way as to the timing or order of any methods of treating cancer when combined with other methods of treating cancer described herein and known by persons of ordinary skill in the art. Any of the foregoing methods of treating cancer can be performed in all conceivable orders and combinations known to one of ordinary skill in the art. For example, a Bcl-2 inhibitor can be administered in combination with surgical therapy wherein the Bcl-2 inhibitor is given prior to, during, or after surgical therapy. The scope of the appended claims is not to be limited to the representative embodiments described herein.

### Kits of the Invention

In certain aspects of the invention there is a kit suitable for use in the invention. In particular embodiments, the invention is drawn to a kit used for determining cancer prognosis, diagnosis, therapeutic target, or pharmacogenetic biomarker. In other embodiments, the kit comprises one or more reagents for the prognosis of cancer in a sample (e.g., tumor biopsy or blood).

Reagents that are suited for obtaining blood or plasma or serum from an individual may be included in a kit of the invention, such as a syringe, collection vial, needle, and so forth, objects known to one of ordinary skill in the art.

The kits may comprise a suitably aliquoted composition and/or additional agent compositions of the present invention, whether labeled or unlabeled, as may be used to prepare a standard curve for a detection assay. The components of the kit may be packaged in combination or alone in the same or in separate containers, depending on, for example, cross-reactivity or stability, and can also be supplied in solid, liquid, lyophilized, or other applicable form. The container means of the kits will generally include, for example, at least one vial, test tube, flask, bottle, syringe or other container means, into which a component may be placed, and preferably, suitably aliquoted. Where there is more than one component in the kit, the kit can contain a second, third or other additional container into which the additional components may be contained. However, various combinations of components may be comprised in a vial. The kits of the present invention also will typically include a means for containing the composition, additional agent, and any other reagent containers in close confinement for commercial sale. Such containers may include, for example, injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In this case, the container means may itself be a syringe, pipette, and/or other such like apparatus, from which the formulation may be applied to an infected area of the body, injected into an animal, and/or even applied to and/or mixed with the other components of the kit.

However, in other embodiments the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the composition is placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained. Irrespective of the number and/or type of containers, the kits of the invention may also comprise, and/or be packaged with, an instrument for assisting with the injection/administration and/or placement of the composition within the body of an animal. Such an instrument may be a syringe, pipette, forceps, and/or any such medically approved delivery vehicle.

### EXAMPLES

### Methods of Prognosis

A novel approach of analyzing gene expression data related to cancer was performed using a publicly available dataset from the Rosetta study linking gene expression profile to disease prognosis in breast cancer patients with primary tumors (van 't Veer et al., Nature, 415:530-536 (2002)). In this study, all patients were under 55 years of age, lymph node negative at the time of diagnosis and sample collection, and poor prognosis was defined as distant metastatic relapse within five years (Id.).

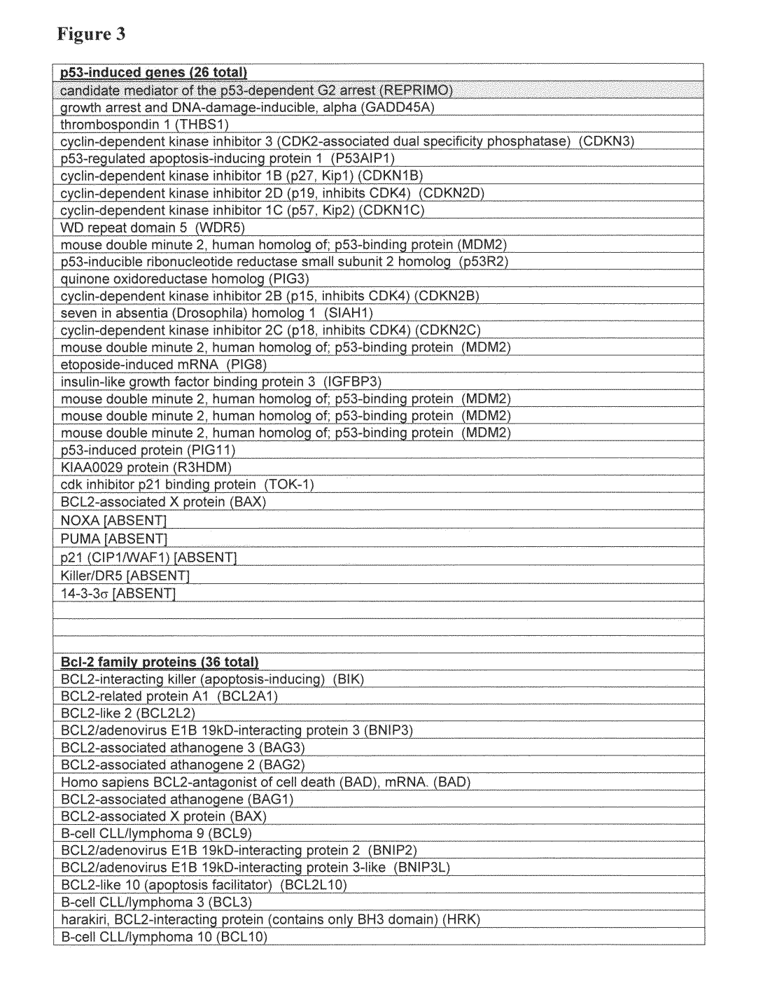

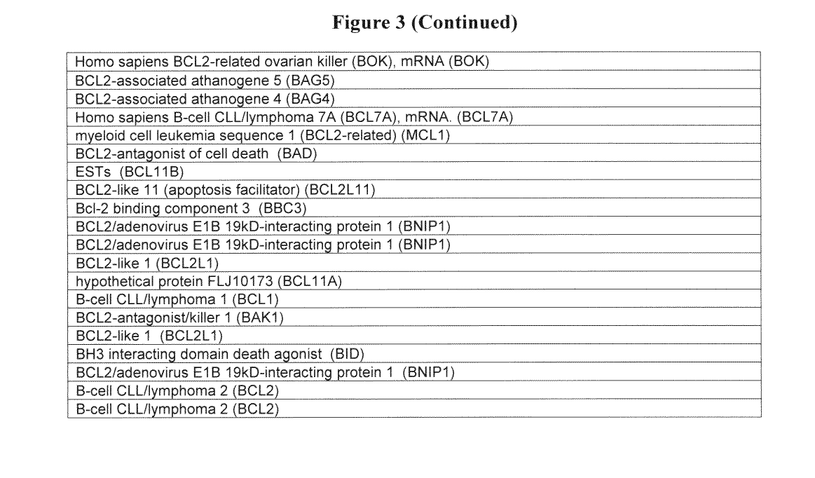

The expression of 26 genes that are known to be p53-inducible (FIG. 3) were analyzed as indicators of intact p53 function. The gene Reprimo indicated intact p53 function across the patient population (49 out of 78 samples with more than two-fold difference from control). Reprimo is a regulator of p53-mediated G2/M arrest and shows some qualities that suggest it could be an effective indicator of p53 function (Ohki et al., J. Biol. Chem., 275:22627-22630 (2000)). First, Reprimo is expressed at a low albeit detectable level in normal cells, but is strongly induced by p53 following DNA damage (Id.). Secondly, cells that are null for p53 show greatly reduced levels of Reprimo, with or without DNA damage (Id.). The GADD45 gene also indicated intact p53 function, significantly altered from control in 24 of the patient samples. Remaining p53-inducible genes were altered in 15 samples or less, or were not present on the array (NOXA, PUMA, p21, Killer/DR5, 14-3-3 sigma). In light of this, Reprimo and GADD45 were used as an indicator of p53 function, although it is contemplated that markers other than Reprimo and GADD45 can be used to indicate p53 function.

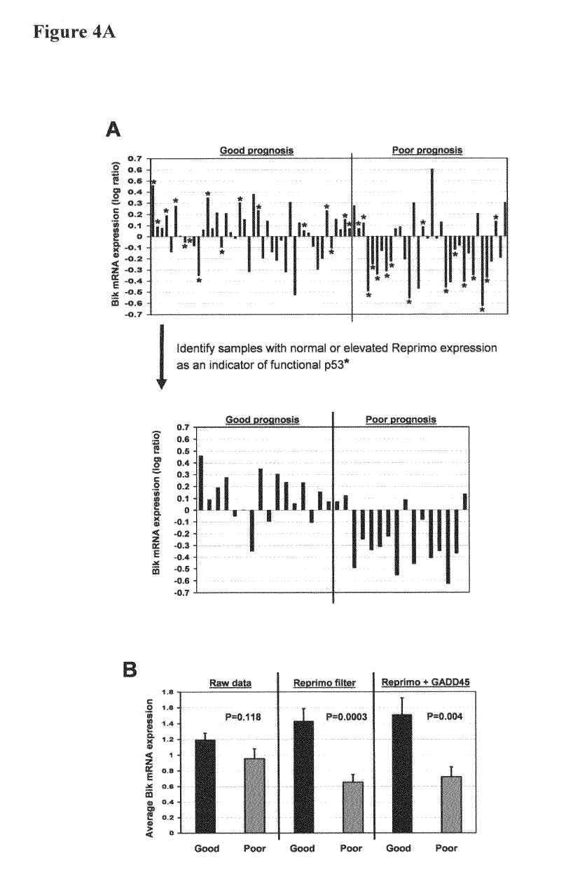

Samples in which Reprimo or GADD45 were downregulated more than two-fold from control, for example, indicated a p53 loss-of-function. Downregulation of Reprimo or GADD45 can be anywhere from about 0.1 times to about 100 times that of a control level. By focusing on the samples that retain intact p53 signaling, the Bik gene was identified as strongly downregulated in breast cancer patients with poor prognosis (FIG. 4). Bik is a pro-apoptotic protein of the Bcl-2 family that sensitizes cells to apoptosis (Martin et al., Biochim. Biophys. Acta., 1692:145-157 (2004)). While a slight correlation between prognosis and Bik expression was apparent from the raw dataset, it was not statistically significant (P=0.118, n=78), which was probably why it was not noted in the original Rosetta study (van 't Veer et al., Nature, 415:530-536 (2002)). Once the dataset is filtered on the basis of Reprimo expression, the statistical significance of reduced Bik expression in patients with poor prognosis becomes apparent (P=0.0003, n=32). Combining Reprimo and GADD45 also identifies the prognostic significance of Bik (P=0.004, n=24). A filter based on GADD45 alone showed the same trend, but did not reach statistical significance (P=0.085, n=59).

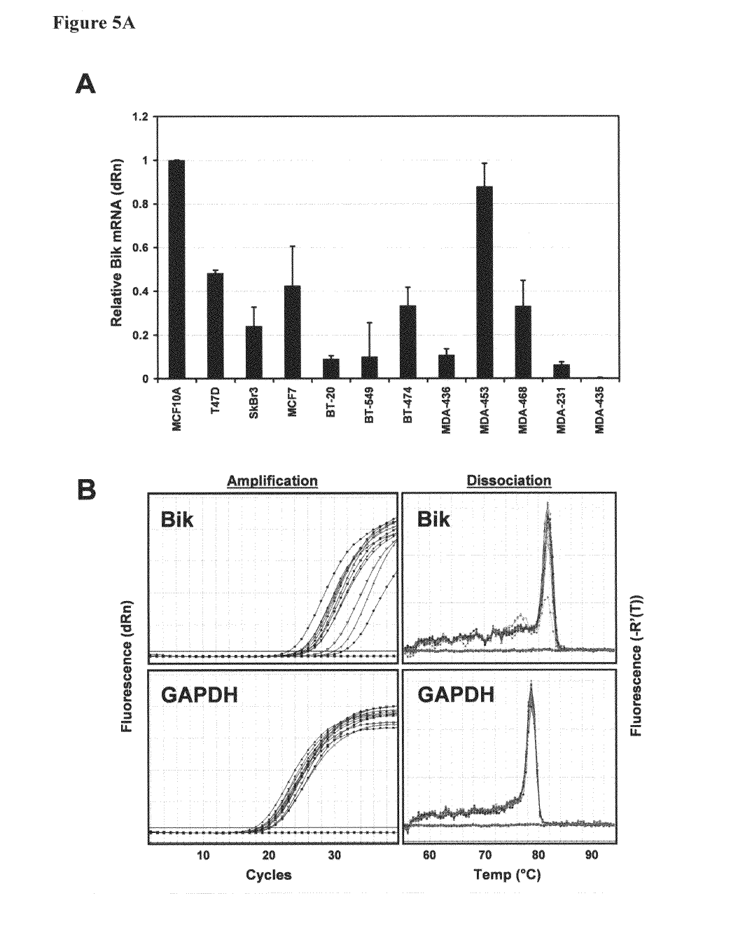

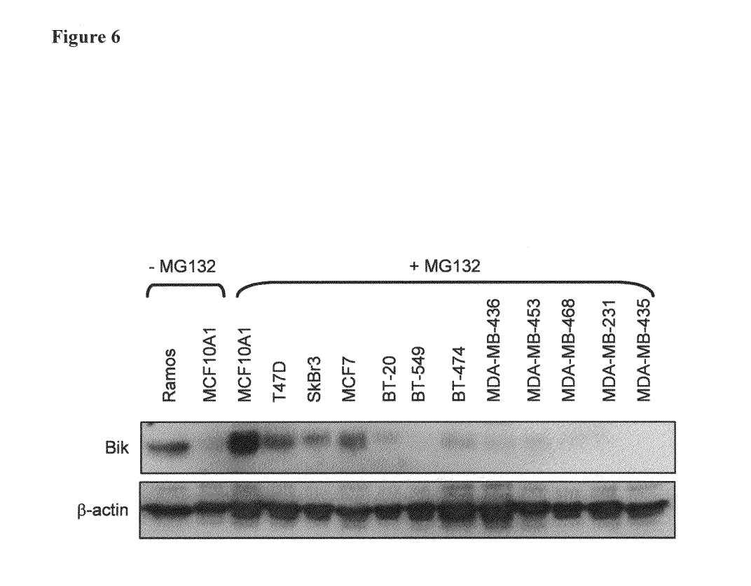

These novel results indicate that loss of Bik expression is a common event in breast tumors and contributes to apoptotic resistance. When assessed by real-time PCR, Bik mRNA was significantly reduced in 10 out of 11 breast tumor cell lines compared to the MCF10A nontumorigenic human mammary epithelial cell line (FIG. 5A). Bik values were normalized to amplification of GAPDH, which was highly consistent in all cell lines and dissociation curves confirmed specificity with a single major amplification product in each reaction type (FIG. 5B). Despite the detectable levels of mRNA, Bik protein levels were very low in the tumor lines and MCF10A cells under normal growth conditions. Since Bik can be degraded by the proteasome (Zhu et al., Oncogene, 24:4993-4999 (2005)), cells were treated with MG132 to inhibit the proteasome and increase detectable Bik levels (FIG. 6). MCF10A cells showed strong induction of Bik protein when degradation was inhibited. All 11 of the tumor cell lines showed significantly lower levels of Bik protein, even with MG132 treatment. Notably, MDA-MB-453 cells had low levels of Bik protein, despite relatively normal levels of Bik mRNA (FIG. 5). Downregulation of Bik in MDA-MB-453 cells therefore occurs independently from mRNA expression. However, loss of Bik function as a common event in breast tumors continues to be supported by these finding.

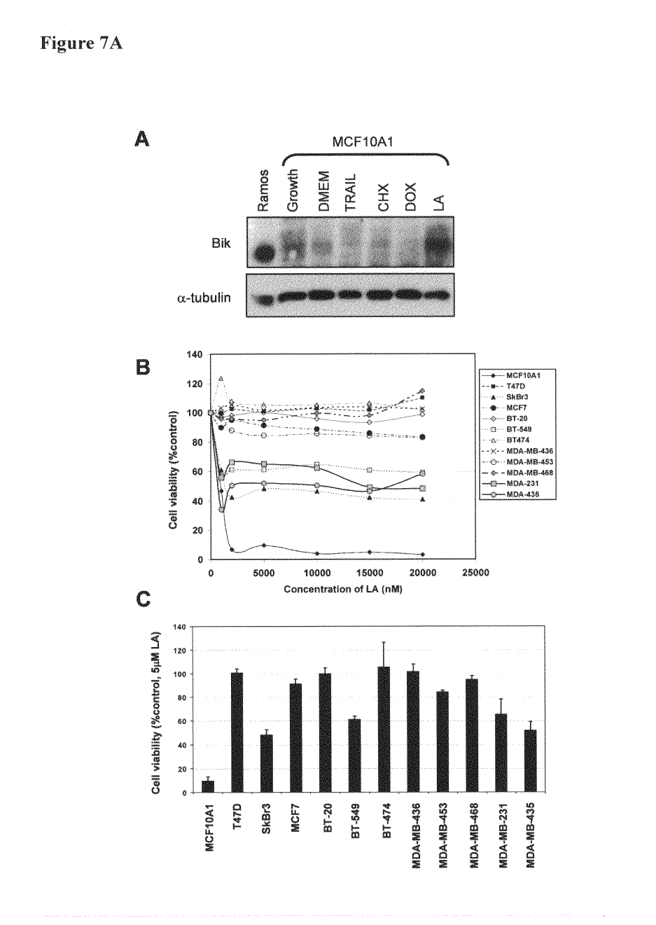

Several apoptotic stimuli were tested for their ability to induce Bik expression in MCF10A cells were evaluated (FIG. 7A). Compared to a Ramos positive control lysate, MCF10A cells express very low levels of Bik under normal growth conditions. Serum starvation in DMEM, treatment with TRAIL, cycloheximide or Doxorubicin did not induce significant expression of Bik, even after 24 hours. Each of these treatments does induce apoptosis in MCF10A cells, as assessed by PARP cleavage. Bik expression in MCF10A cells increased after treatment with Latrunculin-A (LA), an inhibitor of the actin cytoskeleton that causes cell rounding (Martin et al., Mol. Cell. Biol., 21:6529-6536 (2001)). Apoptosis caused by cell rounding and cytoskeletal disruption has been termed amorphosis (Martin et al., Biochim. Biophys. Acta., 1692:145-157 (2004)), and resistance to amorphosis is thought to contribute to metastatic potential by allowing tumor cells to tolerate the cell shape changes that occur during bloodborne dissemination (Martin et al., Oncogene, 23:4641-4645 (2004); Mehlen et al., Nat. Rev. Cancer, 6:449-458 (2006); Pinkas et al., Mol. Cancer. Res., 2:551-556 (2004)). While MCF10A cells are highly sensitive to amorphosis (Martin et al., Mol. Cell. Biol., 21:6529-6536 (2001)), all of the breast tumor cell lines with reduced Bik expression are highly resistant to LA-induced apoptosis, even at very high doses (FIG. 7B). Cell death is maximal after treatment with 5 μM LA in MCF10A cells, but all tumor cell lines demonstrate significant resistance (FIG. 7C) that correlates strongly with their reduced expression of Bik protein. Since Bik is induced by cell rounding, its loss in tumors may reduce their apoptotic sensitivity and yield a greater metastatic potential. In fact, reduced expression of Bik predicts increased nodal involvement in colorectal cancer (Bandres et al., Oncol. Rep., 12:287-292 (2004)). Conversely, forced expression of Bik inhibits systemic breast tumor growth (Zou et al., Cancer Res., 62:8-12 (2002)).

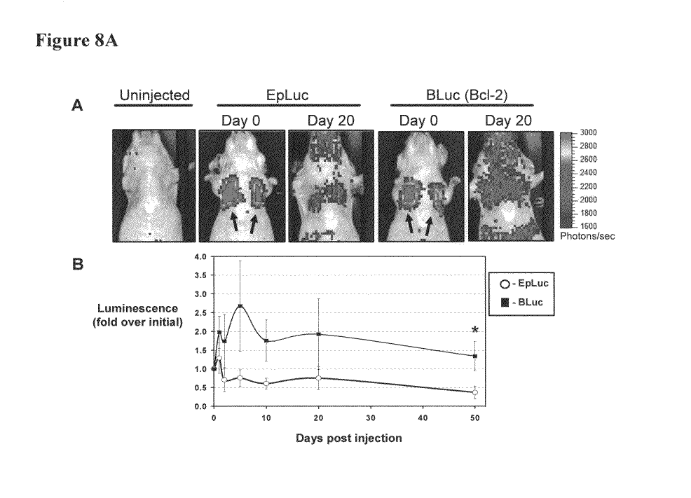

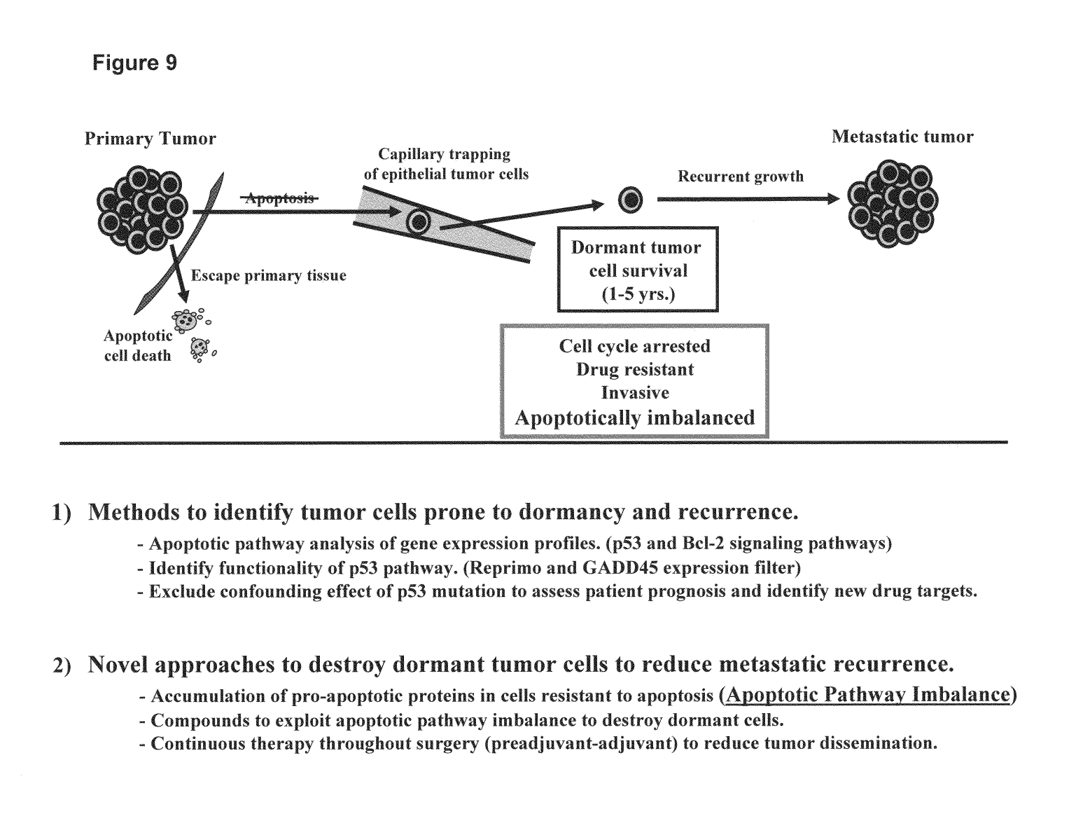

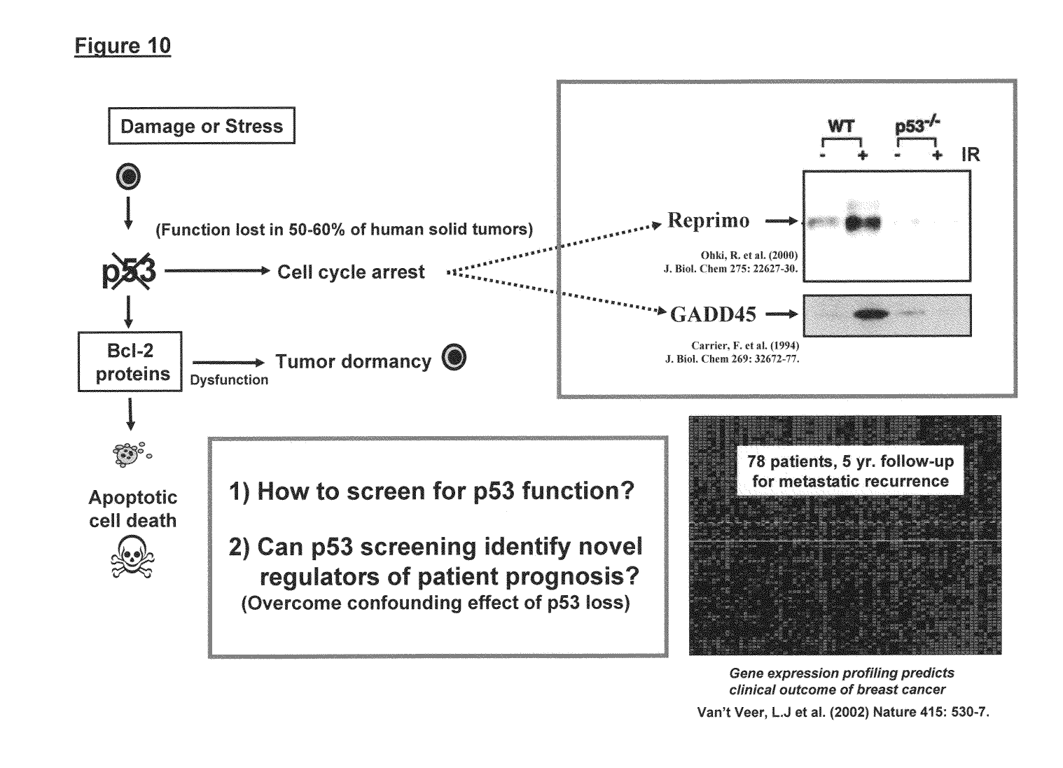

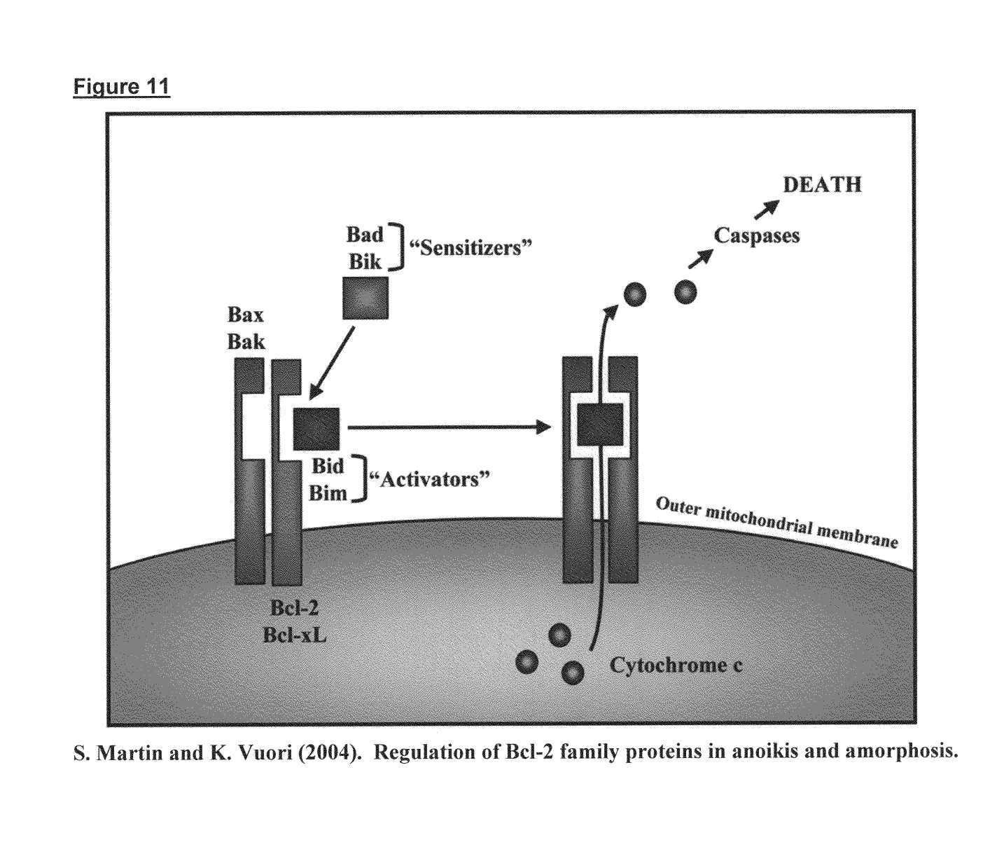

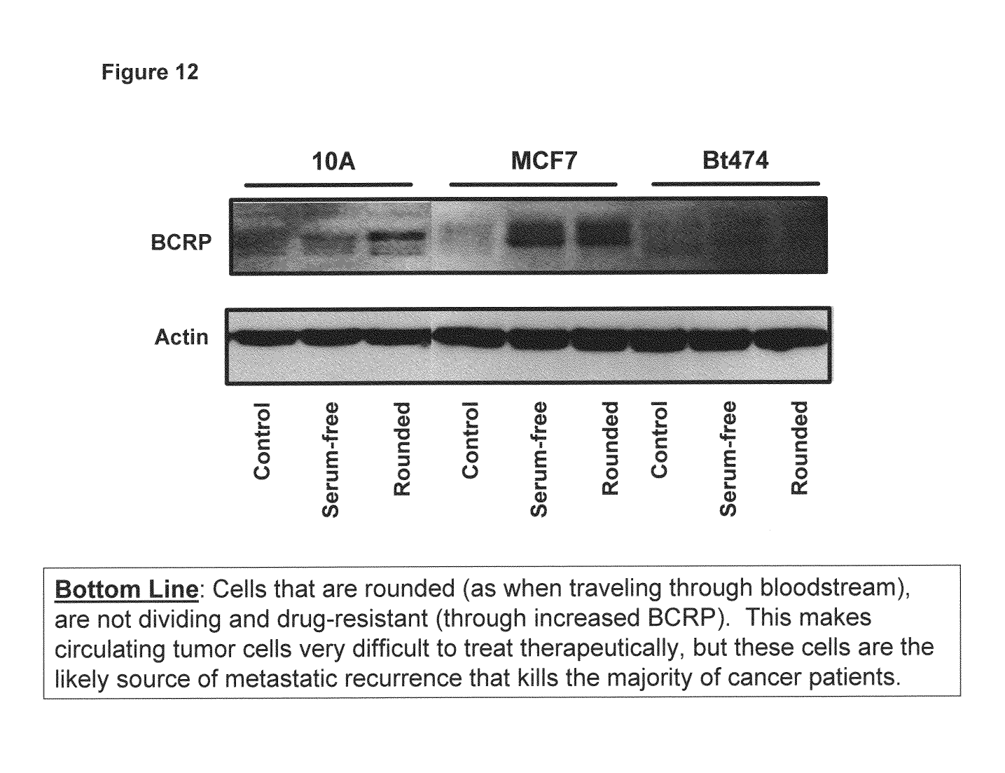

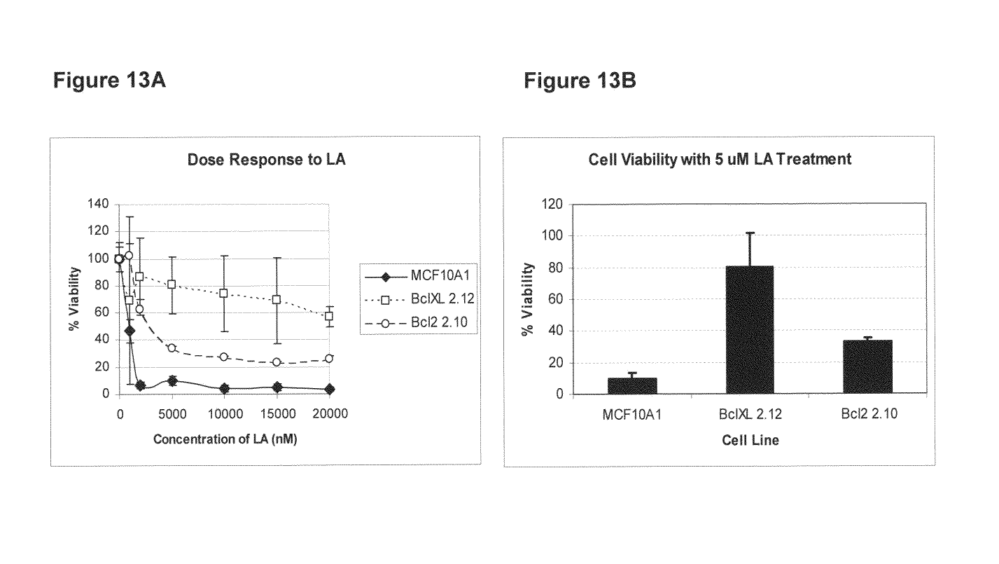

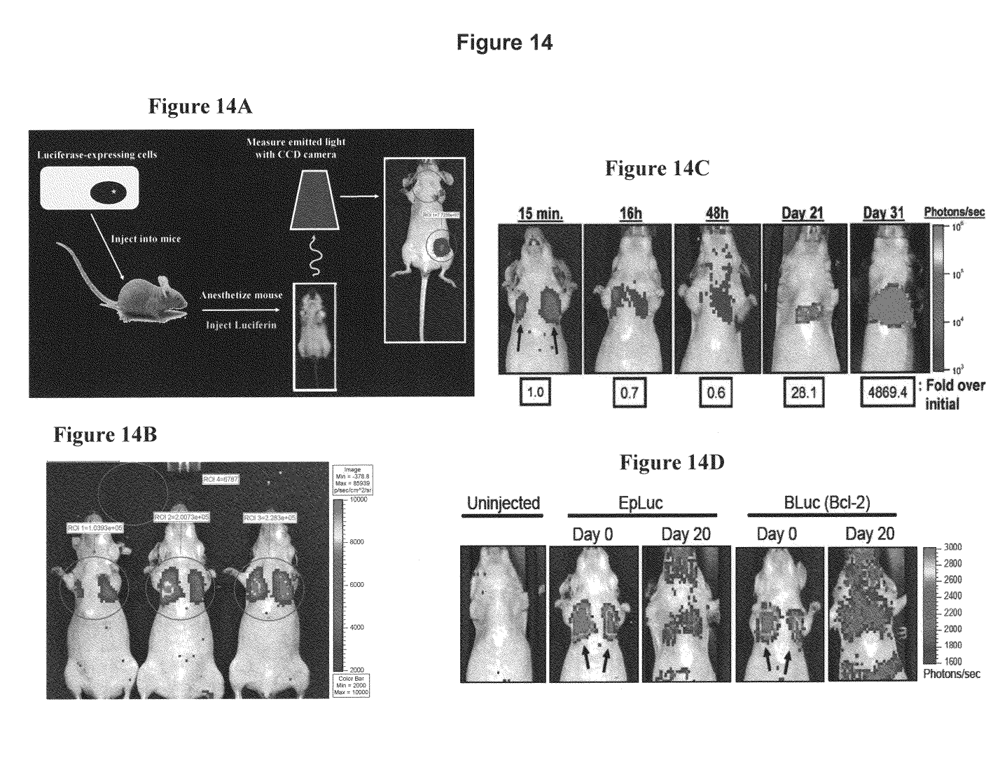

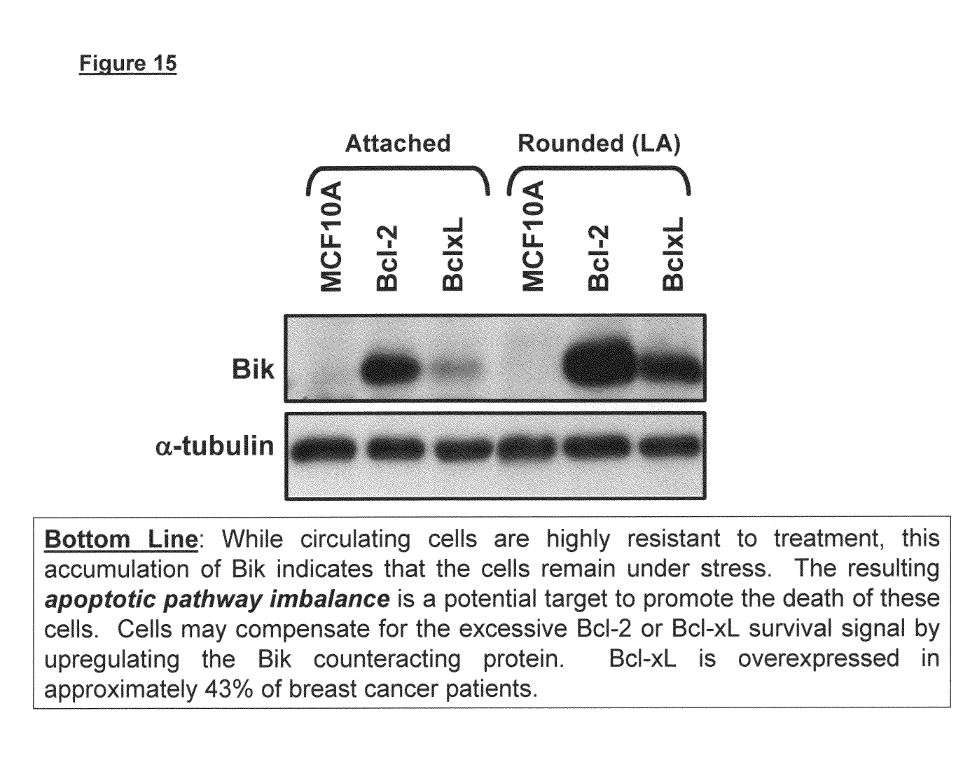

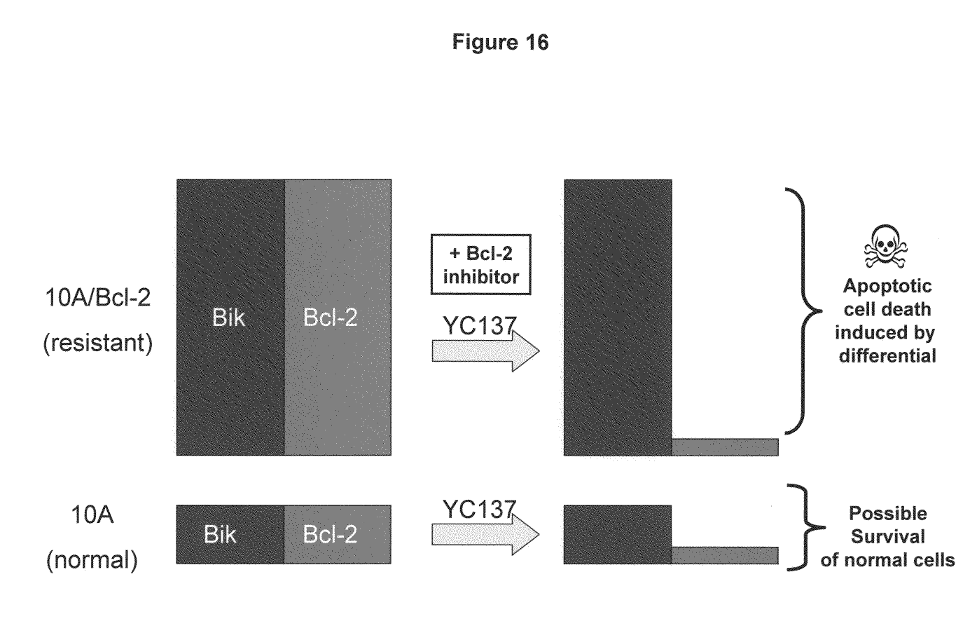

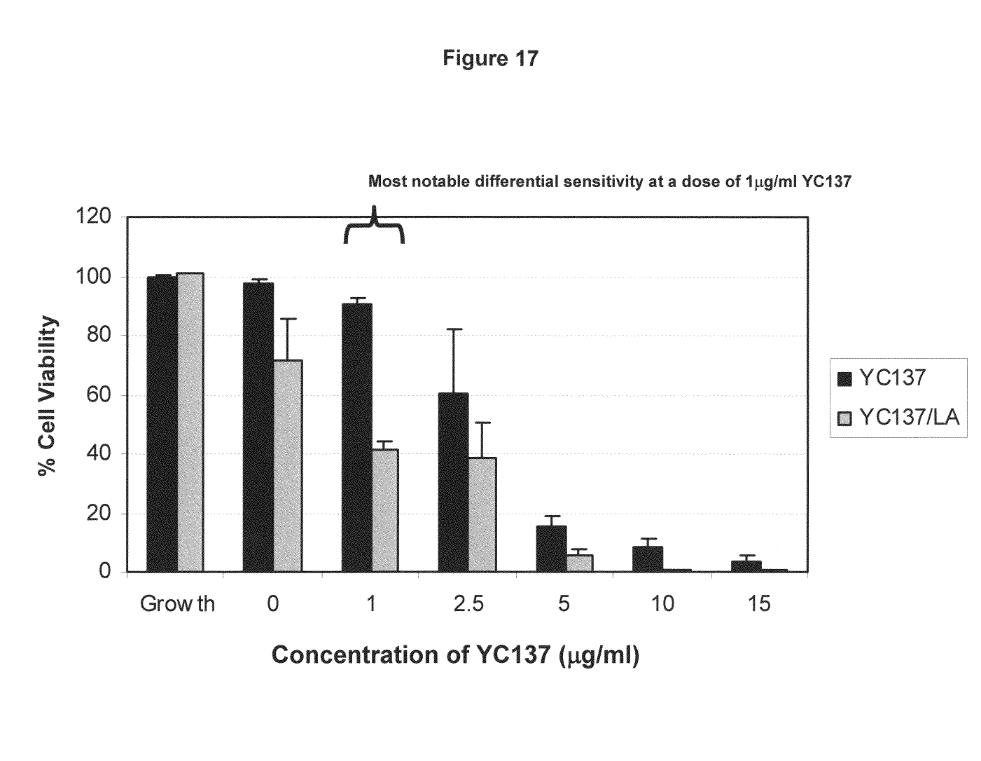

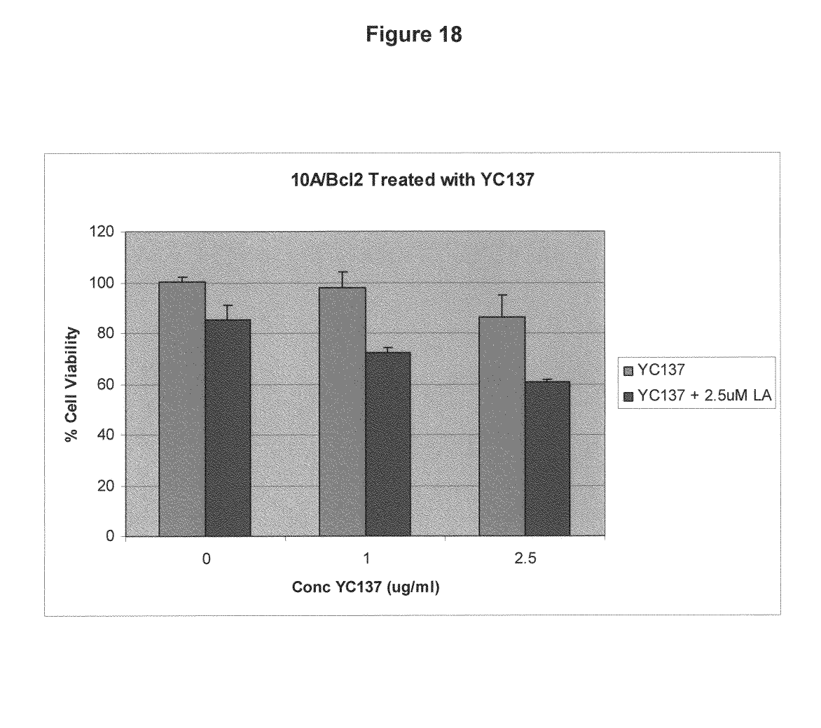

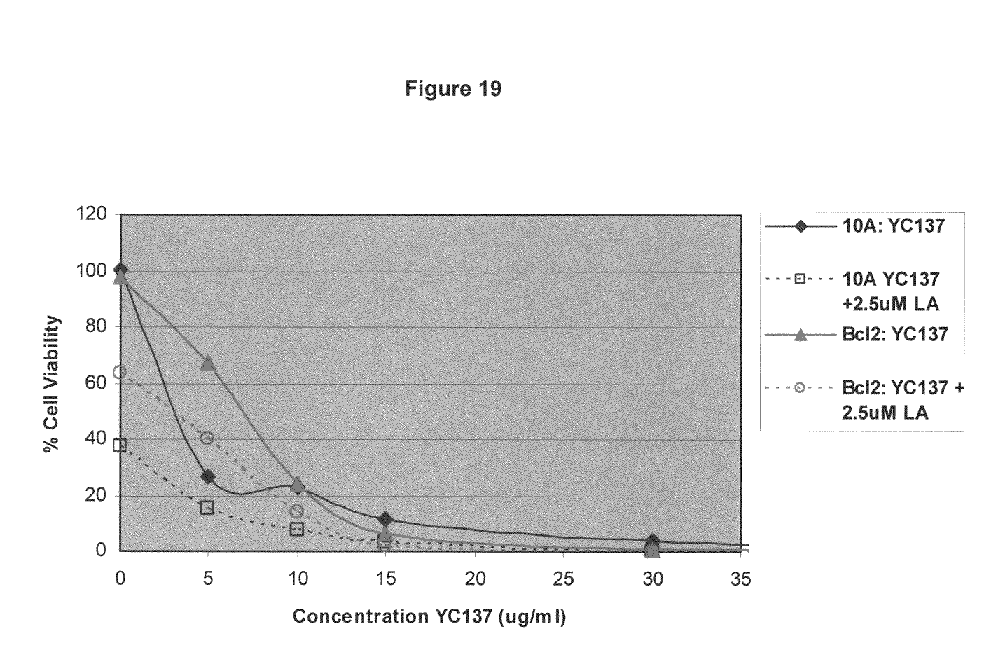

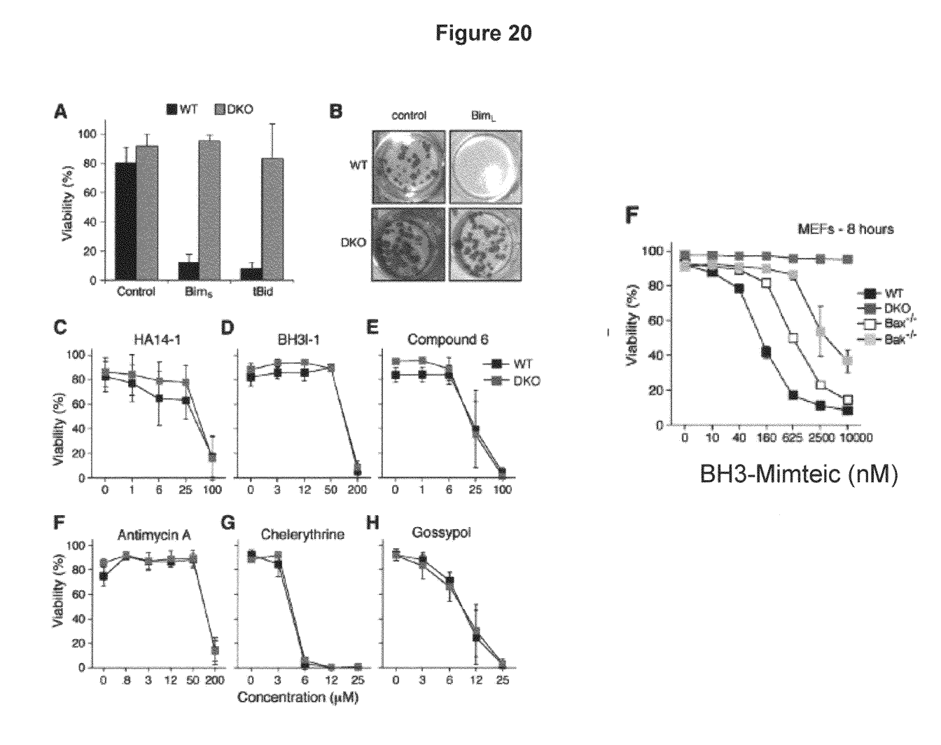

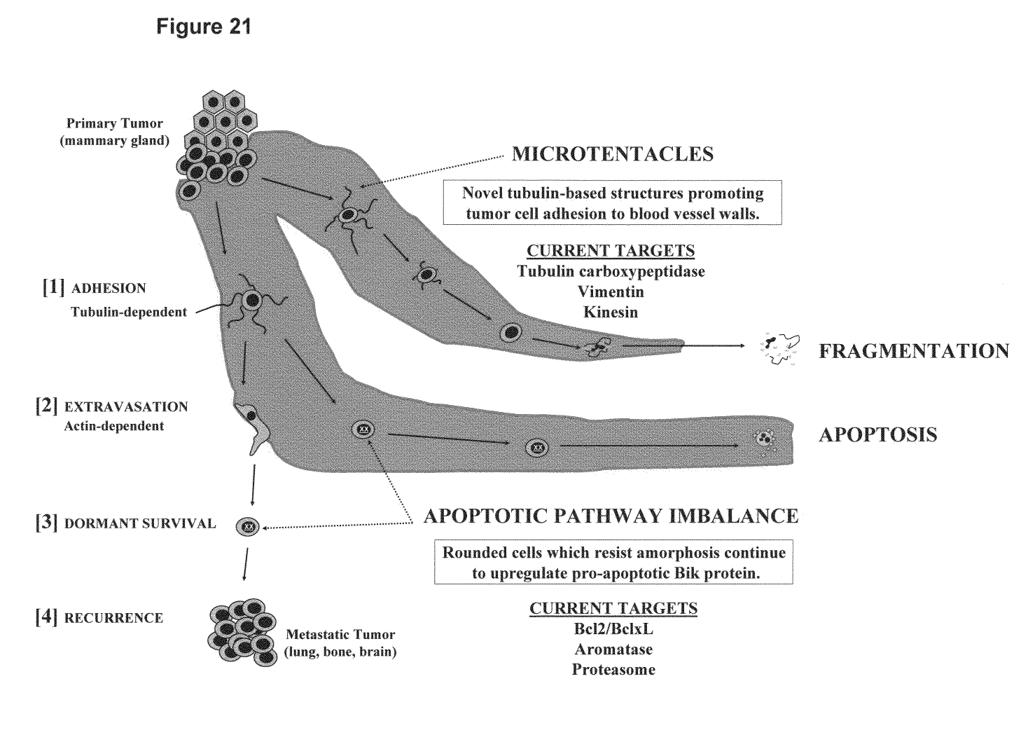

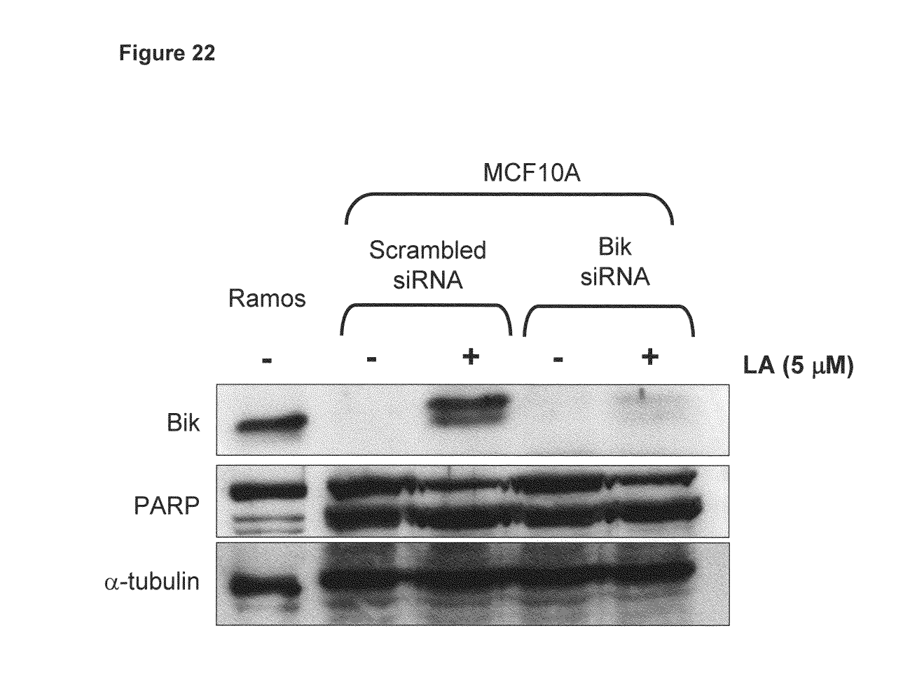

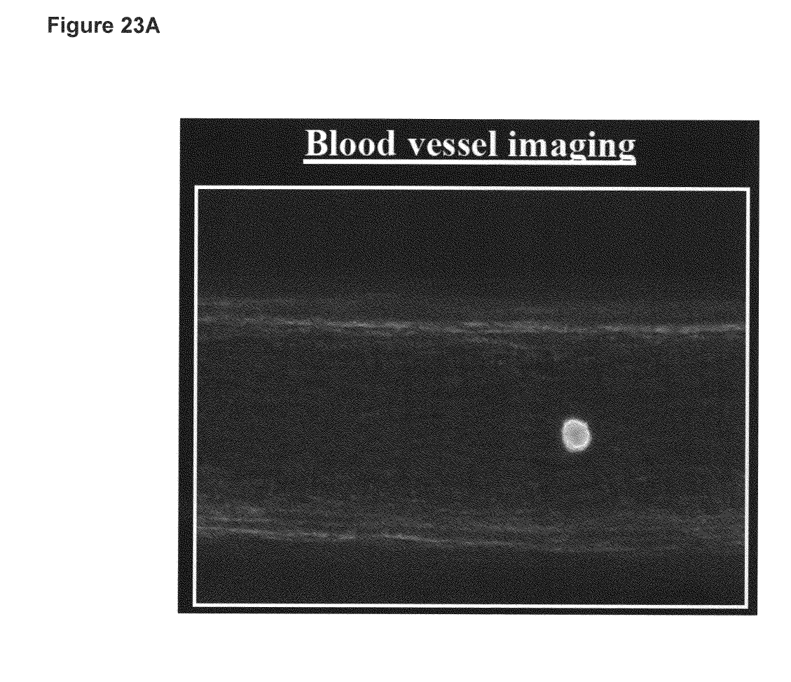

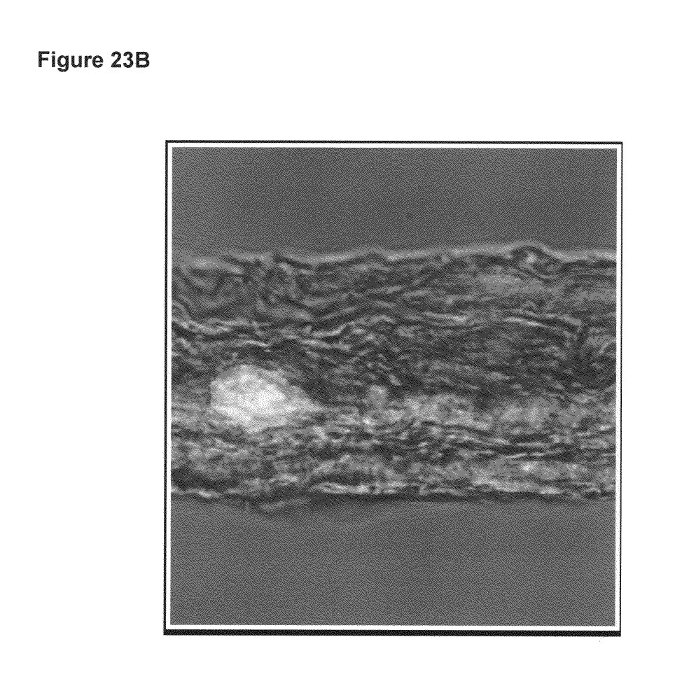

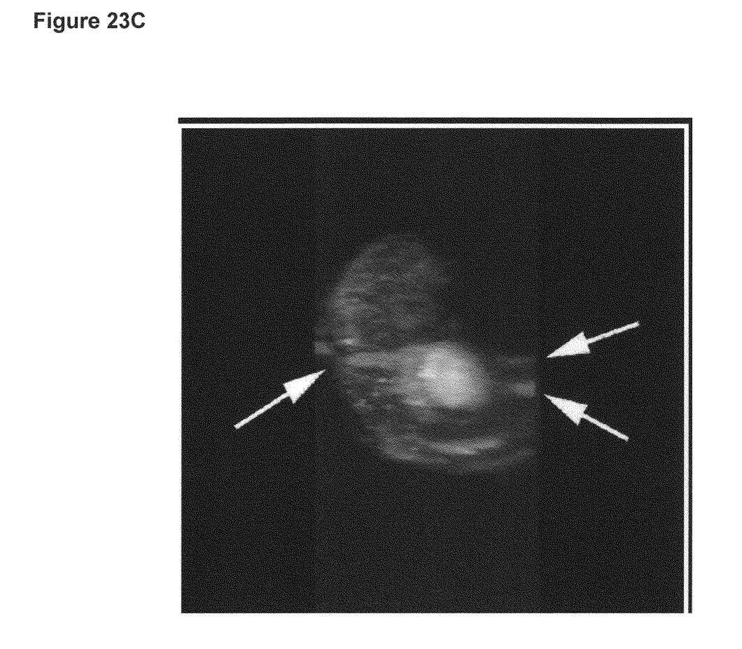

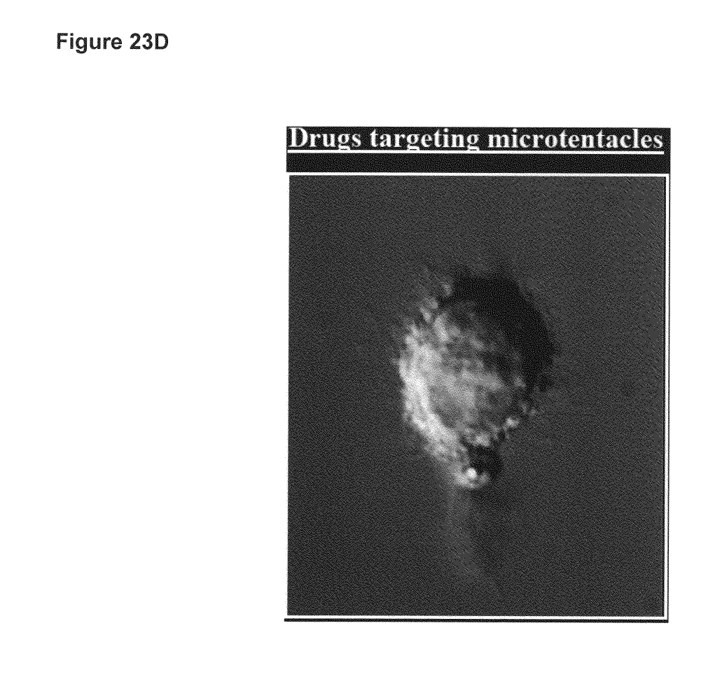

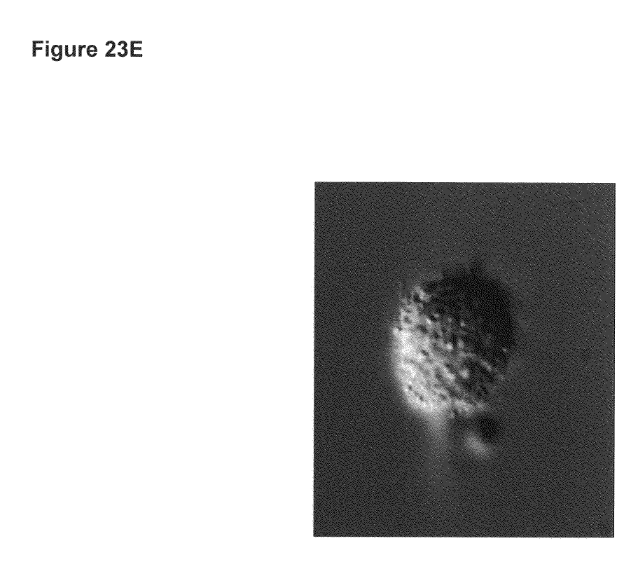

Inhibiting apoptosis with Bcl-2 overexpression can increase cell survival in the bloodstream, but p53 retains its ability to restrict the cell cycle through p21 (Nikiforov et al., Oncogene, 15:3007-3012 (1997) (FIG. 8)). For this reason, apoptotic resistance on its own is not thought sufficient to induce metastatic breast tumor growth (Martin et al., Oncogene, 23:4641-4645 (2004)), and may promote a period of tumor dormancy in which cells survive dissemination but do not grow immediately (Naumov et al., Breast Cancer Res. Treat., 82:199-206 (2003)). Such dormant cells have been shown to be highly resistant to standard chemotherapies due to their lack of active cell cycling (Id.). Overexpression of Bcl-2 can also directly induce cell cycle arrest, but this ability segregates from its anti-apoptotic function during tumor progression (Furth et al., Oncogene, 18:6589-6596 (1999)). This dual nature of apoptotic resistance to inhibit active tumor growth but promote survival may explain the ability of Bcl-2 overexpression to suppress tumor cell proliferation (Id.) but promote metastatic dissemination (Pinkas et al., Mol. Cancer. Res., 2:551-556 (2004)). Loss of Bik expression would be predicted to affect tumors in a similar way as overexpression of Bcl-2 or Bcl-xL, promoting dormancy and dissemination, but not immediate growth (Martin et al., Oncogene, 23:4641-4645 (2004); Pinkas et al., Mol. Cancer. Res., 2:551-556 (2004)). However, the eventual re-emergence of these dormant and disseminated cells has dire consequences for breast tumor patients and could be why reduced Bik expression is associated with poor prognosis.

These findings are the first to show that loss of Bik expression is a common event in breast tumor cell lines and can be associated with more rapid metastatic relapse in breast cancer patients. It is important to note that the significance of this alteration of Bik expression would not have been apparent without first identifying samples with transcriptional evidence of intact p53 signaling. Given the prevalence of p53 loss-of-function in human cancers and its powerful selective advantage, gene expression studies can now account for p53 pathway function when assessing the prognostic significance of apoptosis genes.

The invention teaches how genetic indicators of cancer prognosis are determined when taking into consideration p53 function. The teachings of the application of the invention demonstrate the unexpected result of Bik as a prognostic indicator of breast cancer, which based on the data set used prior to taking into account p53 function, was not a prognostic indicator. Various prognostic indicators associated with various other cancers are included in the methods. This method determines diagnostic markers and targets for cancer therapy.

### REFERENCES

All patents and publications mentioned and/or cited in this specification are evidence of the level of those skilled in the art to which the invention pertains. All patents and publications herein are incorporated by reference to the same extent as if each individual publication was specifically and individually indicated as having been incorporated by reference in its entirety.

What is claimed:

1. A method of treating cancer in a subject in need of such treatment comprising administering to said subject an effective dose of an apoptosis inducing agent wherein said apoptosis inducing agent comprises at least one inhibitor of an anti-apoptotic member of the Bcl-2 family.

2. The method of claim 1, wherein the apoptosis inducing agent is administered in combination with one or more methods of treating cancer selected from the group consisting of administering an anticancer agent, radiation therapy, and surgical therapy.

3. The method of claim 2, wherein the apoptosis inducing agent is administered in combination with surgical therapy.

4. The method of claim 3, wherein the apoptosis inducing agent is administered in advance of said surgical therapy.

5. The method of claim 3, wherein said surgical therapy is surgical tumor resection.

6. The method of claim 1, wherein said apoptosis inducing agent inhibits Bcl-2.

7. The method of claim 1, wherein said apoptosis inducing agent inhibits Bcl-xL.

8. A method of killing a cancer cell comprising contacting said cancer cell with an effective dose of an apoptosis inducing agent wherein said agent comprises at least one inhibitor of an anti-apoptotic member of the Bcl-2 family.

9. The method of claim 8, wherein said cancer cell is contacted with the apoptosis inducing agent in combination with one or more anticancer agents or radiation therapy.

10. A method of preventing tumor metastasis in a subject in need of such prevention comprising administering to said subject an apoptosis inducing agent wherein said agent comprises at least one inhibitor of an anti-apoptotic member of the Bcl-2 family.

11. The method of claim 10, wherein the apoptosis inducing agent is administered in combination with one or more methods of treating cancer selected from the group consisting of administering an anticancer agent, radiation therapy, and surgical therapy.

12. The method of claim 11, wherein the apoptosis inducing agent is administered in combination with surgical therapy.

13. The method of claim 12, wherein the apoptosis inducing agent is administered in advance of said surgical therapy.

14. The method of claim 11, wherein said surgical therapy is surgical tumor resection.

15. (canceled)

16. (canceled)

17. (canceled)

18. A method of treating cancer in a subject in need of such treatment comprising administering to said subject an effective dose of at least one apoptosis inducing agent, wherein said agent is selected from the group consisting of Bak, Bmf, Bik, Bid, Bad, Bim, Bcl-2, and Bok, Bax, Mctl, Hrk, Noxa, PUMA and Bcl-x, or combinations thereof.

19. The method of claim 18, further comprising regulating apoptotic pathways.

20. The method of claim 18, further comprising regulating an apoptotic pathway wherein said apoptotic pathway includes Mcl-1, A-1 (Bfl-1), Boo, NR-13, and Bcl-W.

21. (canceled)

22. (canceled)

23. (canceled)
